(19) United States
(12) Patent Application Publication
(10) Pub. No.: US 2004/0146949 A1
(43) Pub. Date: Jul. 29, 2004

(54) Methods and compounds for disruption of CD40R/CD40L signaling in the treatment of alzheimer's disease

(76) Inventors: Jun Tan, Bradenton, FL (US); Terrence C. Town, Bradenton, FL (US); Michael Mullan, Bradenton, FL (US)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/694,634

(22) Filed: Oct. 27, 2003

Related U.S. Application Data
(60) Provisional application No. 60/421,338, filed on Oct. 25, 2002.

(51) Int. Cl. G01N 33/53; G01N 33/567

(52) U.S. Cl. 435/7.2; 800/3

(57) ABSTRACT

The present invention provides a research model for screening compounds suspected of modulating the CD40L/CD40R signaling pathway by interfering with the CD40L/CD40R signaling pathway in an animal or human. Additionally, methods are provided for causing a desired biological effect in an individual or system afflicted with neuronal inflammation, brain injury/trauma, a tauopathy, or an amyloidogenic disease, as well as for the identification of compounds and/or small molecules capable of disrupting the CD40L/CD40R signaling pathway.

### CROSS REFERENCE TO RELATED APPLICATION

The present invention claims priority to U.S. Provisional Application Serial No. 60/421,338, filed Oct. 25, 2002.

### BACKGROUND OF THE INVENTION

1. Field of the Invention

This invention relates generally to methods and compositions for use in the treatment of Alzheimer's and related amyloidogenic diseases, and to methods for screening such compounds. More specifically, this invention relates to methods and/or assay systems for the identification of compounds or other small molecules capable of disrupting the CD40 receptor/CD40 ligand (CD40R/CD40L) signaling pathway in an animal or human afflicted with an amyloidogenic disease.

2. Description of Related Art

Deposition of β-amyloid in mammalian brain is a defining feature of Alzheimer's disease, and there is evidence that activation of inflammatory pathways is important in the pathogenesis of the disease. With age, transgenic mice that overexpress the “Swedish” mutant amyloid precursor protein (Tg APP_{sw}, line 2576), show markedly elevated levels of cortical deposited β-amyloid (Aβ) and gliosis. The CD40 receptor (CD40R) is a key immunoregulatory molecule, and we have shown that pro-inflammatory microglial activation which is induced by Aβ peptides requires the ligation of CD40R with its cognate ligand CD40L.

Alzheimer's disease (AD) is a progressive neurodegenerative disease that afflicts approximately 1% of the population over the age of 65. Characteristic features of the disease include neurofibrillary tangles composed of abnormal tau protein paired helical filaments, neuronal loss, and alteration in multiple neurotransmitter systems. A significant pathological feature is an overabundance of diffuse and compact senile plaques in association with limbic areas of the brain. Although these plaques contain multiple proteins, their cores are composed primarily of Aβ, a 39-42 amino acid proteolytic fragment derived from amyloid precursor protein (APP).

Alzheimer's disease is not usually inherited but genes do play a role in a proportion of cases. Three genes have been identified that, if defective, cause Alzheimer's disease. All the disease-causing mutations alter the processing of APP in such a way that they increase Aβ₁₋₄₂ accumulation. The affected genes that encode APP are located on chromosome 21. Individuals with Downs Syndrome (which results from partial or complete trisomy of chromosome 21) also develop plaques and tangles in the brain by their 40's. Five mutations have been identified on chromosome 21 associated with Alzheimer's disease. Another gene, presenilin-1 located on chromosome 14, is associated with Alzheimer's disease. Presenilin-1 controls presenilin protein expression which in turn alters Aβ formation. Mutation of this gene increases Aβ levels and may account for approximately 50% of early-onset Alzheimer's disease. The presenilin-2 gene, located on chromosome 1, encodes for a similar protein as presenilin-1 with similar effects on APP processing. Mutations of this gene may account for approximately 10% of familial Alzheimer cases.

APP is a single-transmembrane protein with a 590-680 amino acid extracellular amino terminal domain and an approximately 55 amino acid cytoplasmic tail. Messenger RNA from the APP gene on chromosome 21 undergoes alternative splicing to yield eight possible isoforms, three of which (the 695, 751 and 770 amino acid isoforms) predominate in the brain. APP undergoes proteolytic processing via three enzymatic activities, termed α-, β- and γ-secretase. Alpha-secretase cleaves APP at amino acid 17 of the Aβ domain, thus releasing the large soluble amino-terminal fragment α-APP for secretion. Because α-secretase cleaves within the Aβ domain, this cleavage precludes Aβ formation. Alternatively, APP can be cleaved by β-secretase to define the amino terminus of Aβ and to generate the soluble amino-terminal fragment β-APP. Subsequent cleavage of the intracellular carboxy-terminal domain of APP by γ-secretase results in the generation of multiple peptides, the two most common being 40-amino acid Aβ (Aβ40) and 42-amino acid Aβ (Aβ42). Aβ40 comprises 90-95% of the secreted Aβ and is the predominant species recovered from cerebrospinal fluid (Seubert et al., “Isolation and quantification of soluble Alzheimer's β-peptide from biological fluids,” Nature (1992) 359:325-7). In contrast, less than 10% of secreted Aβ is Aβ42. Despite the relative paucity of Aβ42 production, Aβ42 is the predominant species found in plaques and is deposited initially (Iwatsubo et al., “Visualization of Aβ42(43) and Aβ40 in senile plaques with specific Aβ monoclonals: evidence that the initially deposited species is Aβ42(43),” Neuron (1993) 13:45-53), perhaps due to its ability to form insoluble amyloid aggregates more rapidly than Aβ40 (Jarrett et al., “The carboxy terminus of β-amyloid protein is critical for the seeding of amyloid formation: Implications for pathogenesis of Alzheimer's disease,” Biochemistry (1993) 32:4693-7; Jarrett et al., “Seeding ‘one-dimensional crystallization’ of amyloid: a pathogenic mechanism in Alzheimer's disease and scrapie?” Cell (1993) 73:1055-8).

Concomitant with Aβ deposition, there exists robust activation of inflammatory pathways in AD brain, including production of pro-inflammatory cytokines and acute-phase reactants in and around Aβ deposits (McGeer et al., “Inflammation in the brain in Alzheimer's disease: Implications for therapy,” J. Leukocyte Biol. (1999) 65:409-15; McGeer et al., “The importance of inflammatory mechanisms in Alzheimer's disease,” Exp. Gerontol. (1998) 33:371-8; Rogers et al., “Inflammation and Alzheimer's disease pathogenesis,” Neurobiol. Aging (1996) 17:681-6). Activation of the brain's resident innate immune cells, the microglia, is thought to be intimately involved in this inflammatory cascade, as reactive microglia produce pro-inflammatory cytokines, such as tumor necrosis factor alpha (TNF-α) and interleuking-1β, which at high levels promote neurodegeneration (Rogers et al., “Inflammation and Alzheimer's disease pathogenesis,” Neurobiol. Aging (1996) 17:681-6; Meda et al., “Activation of microglial cells by beta-amyloid and interferon-gamma,” Nature (1995) 374:647-50; Barger et al., “Microglial activation by Alzheimer amyloid precursor protein and modulation by apolipoprotein E,” Nature (1997) 388:878-81). Epidemiological studies have shown that patients using non-steroidal anti-inflammatory drugs (NSAIDS) have as much as a 50% reduced risk for AD (Rogers et al., “Inflammation and Alzheimer's disease pathogenesis,” Neurobiol. Aging (1996) 17:681-6; Stewart et al., “Risk of Alzheimer's disease and duration of NSAID use,” Neurology (1997) 48:626-32), and post-mortem evaluation of AD patients who underwent NSAID treatment has demonstrated that risk reduction is associated with diminished numbers of activated microglia (Mackenzie et al., “Nonsteroidal anti-inflammatory drug use and Alzheimer-type pathology in aging,” Neurology (1998) 50:986-90). Further, when Tg APP_{sw} mice are given an NSAID (ibuprofen), these animals show reduction in Aβ deposits, astrocytosis, and dystrophic neurites correlating with decreased microglial activation (Lim et al., “Ibuprofen suppresses plaque pathology and inflammation in a transgenic mouse model for Alzheimer's disease,” J. Neurosci. (2000) 20:5709-14).

Recent studies, however, have indicated that the relationship between microglial activation and promotion of AD-like pathology is not straightforward, as some forms of microglial activation appear to mitigate this pathology. Schenk et al. have shown that immunization of PDAPP mice (a transgenic mouse model of AD which overexpresses APP) with Aβ42 results in a marked reduction of Aβ deposits, and atypical punctate structures containing Aβ, which resemble activated microglia, were found in brains of these mice, suggesting that immunization activates microglia to phagocytose Aβ (Schenk et al., “Immunization with beta-amyloid attenuates Alzheimer-disease-like pathology in the PDAPP mouse,” Nature (1999) 400:173-7). This hypothesis was further supported ex vivo, where microglia were shown to clear deposited Aβ that was opsonized by anti-Aβ antibodies (Bard et al., “Peripherally administered antibodies against amyloid beta-peptide enter the central nervous system and reduce pathology in a mouse model of Alzheimer's disease,” Nat. Med. (2000) 6:916-19). Similar prophylactic effects of Aβ42 immunization have now been independently observed in other transgenic mouse models of AD (Morgan et al., “A beta peptide vaccination prevents memory loss in an animal model of Alzheimer's disease,” Nature (2000) 408:982-5; Janus et al., “A beta peptide immunization reduces behavioral impairment and plaques in a model of Alzheimer's disease,” Nature (2000) 408:979-82), and in vivo visualization has shown that administration of anti-Aβ antibody to PDAPP mouse brain results in rapid Aβ plaque clearance associated with marked local microglial activation (as measured by lectin immunoreactivity) (Bacskai et al., “Imaging of amyloid-beta deposits in brains of living mice permits direct observation of clearance of plaques with immunotherapy,” Nat. Med. (2001) 7:369-72). Finally, bigenic mice that overexpress human APP and transforming growth factor β1 also demonstrate reduced parenchymal Aβ deposition associated with an increase in microglia positive for the F4/80 antigen (Wyss-Coray et al., “TGF-beta1 promotes microglial amyloid-beta clearance and reduces plaque burden in transgenic mice,” Nat. Med. (2001) 7:612-18).

The CD40 receptor is a ˜45 kDa key immunoregulatory molecule belonging to the tumor necrosis factor (TNF) receptor family and plays a critical role in immune cell activation. Signal transduction through CD40R is initiated by binding trimeric CD40L on the surface of activated T cells (Foy et al., Annu. Rev. Immunol., (1996) 14:591-617). Activation of CD40R-dependent signaling pathways is thought to be mediated primarily by recruitment of several TRAF protein family members to the multimerized CD40 cytoplasmic domain (Arch et al., Genes Dev. (1998) 12:2821-2830). The 62-amino acid human CD40 cytoplasmic domain (CD40c) contains two linear TRAF binding sites, a membrane proximal site that binds TRAF6 and a membrane distal site that directly binds TRAF1, TRAF2, and TRAF3 (Pullen et al., Biochemistry (1998) 37:11836-11845). It is believed that CD40R forms at least a trimeric complex upon binding its ligand. Biochemical experiments suggest that the requirement for CD40Rc trimerization in the recruitment of TRAF proteins is avidity-driven. As an alternative to dimerization, receptor trimerization may regulate initiation of CD40R signaling by providing a higher degree of discrimination between liganded and unliganded receptors (Ni et al., Procedure. Natl. Acad. Sci. USA (2000) 10395-10399).

In the periphery, ligation of B cell CD40R promotes B cell proliferation after antigenic challenge, resulting in differentiation into antibody-secreting plasma cells. Blockade of the CD40R/CD40L interaction in vivo inhibits activated T cell-dependent interleukin-12 secretion by antigen presenting cells (Grewal et al., “Requirement for CD40 ligand in costimulation induction, T cell activation, and experimental allergic encephalomyelitis,” Science (1996) 273:1864-7; Stuber et al., “Blocking the CD40L-CD40 interaction in vivo specifically prevents the priming of T helper 1 cells through the inhibition of interleukin 12 secretion,” J. Exp. Med. (1996) 183:693-8).

We and others have shown that CD40 is expressed on cultured microglia at low levels, and CD40R expression is markedly enhanced on these cells by the pro-inflammatory cytokine interferon-γ as well as Aβ (Carson et al., “Mature microglia resemble immature antigen-presenting cells,” Glia (1998) 22:72-85; Tan et al., “Activation of microglial cells by the CD40 pathway: relevance to multiple sclerosis,” J. Neuroimmunol. (1999) 97:77-85; Tan et al., “Microglial activation resulting from CD40-CD40L interaction stimulate microglia to secrete TNF-α, resulting in induction of neuronal injury in vitro, effects that are not observed in the presence of low levels of Aβ alone (Tan et al., “Microglial activation resulting from CD40R-CD40L interaction after beta-amyloid stimulation,” Science (1999) 286:2352-55). Further, interruption of CD40R-CD40L signaling in Tg APP_{sw} mice mitigates hyperphosphorylation of the microtubule-associated protein tau (Tan et al., “Microglial activation resulting from CD40R/CD40L interaction after beta-amyloid stimulation,” Science (1999) 286:2352-55), a known marker of the pathogenic neuronal pre-tangle stage in AD brain. Additionally, in AD brain, CD40R expression is markedly increased on activated microglia and in senile plaques (Togo et al., “Expression of CD40 in the brain of Alzheimer's disease and other neurological diseases,” Brain Res. (2000) 885:117-21). Recently, expression of CD40L and CD40R has been found in and around β-amyloid plaques in AD brain (Calingasan et al., “Identification of CD40 ligand in Alzheimer's disease and in animal models of Alzheimer's disease and brain injury,” Neurobiol. Aging (2002) 23:31-9; Togo et al., “Expression of CD40 in the brain of Alzheimer's disease and other neurological diseases,” Brain Res. (2000) 885:117-21).

There is mounting evidence that products of the inflammatory process in AD brain exacerbate AD pathology. Many of these inflammatory proteins and acute phase reactants, such as alpha-1-antichymotrypsin, transforming growth factor β, apolipoprotein E and complement factors, are produced by activated glia, are localized to Aβ plaques, and have been shown to promote Aβ plaque “condensation” or maturation (Nilsson et al., “Alpha-1-antichymotrypsin promotes beta-sheet amyloid plaque deposition in a transgenic mouse model of Alzheimer's disease,” J. Neurosci. (2001) 21:1444-51; Harris-White et al., “Effects of transforming growth factor-beta (isoforms 1-3) on amyloid-beta deposition, inflammation, and cell targeting in organotypic hippocampal slice cultures,” J. Neurosci. (1998) 18:1366-74; Styren et al., “Expression of differential immune factors in temporal cortex and cerebellum: the role of alpha-1-antichymotrypsin, apolipoprotein E, and reactive glia in the progression of Alzheimer's disease,” J. Comp. Neurol. (1998) 396:511-20; Rozemuller et al., “A4 protein in Alzheimer's disease: primary and secondary cellular events in extracellular amyloid deposition,” J. Neuropathol. Exp. Neurol. (1989) 48:674-91). Further, there is evidence that activated microglia in AD brain, instead of clearing Aβ, are pathogenic by promoting Aβ firbrillogenesis and consequent deposition as senile plaques (Frackowiak et al., “Ultrastructure of the microglia that phagocytose amyloid and the microglia that produce beta-amyloid fibrils,” Acta Neuropathol. (Berl.) (1992) 84:225-33; Wegiel et al., “Microglia cells are the driving force in fibrillar plaque formation, whereas astrocytes are a leading factor in plaque degradation,” Acta Neuropathol. (Berl.) (2000) 100:356-64).

### BRIEF SUMMARY OF THE INVENTION

The present invention provides methods of treating neuronal inflammation, brain injury, brain trauma, tauopathies, or amyloidogenic diseases, via the administration of therapeutically effective amounts of a composition comprised of an agent and a carrier which interferes with the interaction of CD40L and CD40R to an individual afflicted with an amyloidogenic disease. Also provided are methods and/or assay systems for the identification of compounds or other small molecules capable of disrupting the CD40R/CD40L signaling pathway. Compounds may modulate the CD40R/CD40L signaling pathway either by interfering with the association of CD40L and CD40R, by interfering with components of the signaling pathway upstream or downstream of the CD40L/CD40R interaction, or by interfering with the trimerization of CD40R. In one aspect of the invention, compounds or small molecules that interfere with TRAFS are contemplated.

In various embodiments, the cell samples are obtained or derived from the central nervous system (CNS), e.g., biopsied materials obtained from humans, animal models, or peripheral sources. Animal models may be transgenic or non-transgenic, and non-limiting examples of these models include mice, worms, or flies. Cells obtained from these animal models can be immortalized and cultured as cell lines. Additionally, cell samples can include immortalized and non-immortalized cell lines derived from human, higher primate, primate, or murine sources.

The present invention also provides a method for determining the ability of a compound to modulate the CD40L/CD40R signaling pathway by interfering with CD40L/CD40R signaling. Compounds capable of interfering with the CD40L/CD40R signaling pathway include stimulators and inhibitors of the CD40L/CD40R signaling pathway, such as, without limitation, agonistic or antagonistic antibodies. Alternatively, the ability of a compound to modulate CD40L/CD40R interactions can be determined by contacting CD40R and CD40L with the compound and measuring the binding of CD40R with CD40L. In these types of assays, compounds can bind either to CD40L or CD40R. The compounds tested can include, without limitation, small molecules or antibodies specific for CD40L or CD40R.

In various embodiments, methods are provided for measuring the levels of various markers, or combination of markers, associated with the inflammatory response, by measuring the levels of one or more markers. Examples of markers include, without limitation, cytokine markers, such as tumor necrosis factor, interleukin 1, interleukin 6, interleukin 12, interleukin 18, macrophage inflammatory protein, macrophage chemoattractant protein, granulocyte-macrophage colony stimulating factor, macrophage colony stimulating factor, or various combinations thereof. Other markers can include, without limitation, glutamate release, nitric oxide production, nitric oxide synthase, superoxide, superoxide dismutase, or various combinations thereof. Also provided are methods for measuring major histocompatability complex molecules, CD45, CD11b, integrins, or cell surface molecules as markers for the inflammatory response. Also provided are methods for measuring levels, amounts, or deposition of proteins on cells. Examples of proteins that can be measured include, without limitation, Aβ, β-APP, a fragment of β-APP, or combinations thereof.

The present invention further provides a method for conducting in vivo assays of compounds or agents capable of modulating the CD40L/CD40R signaling pathway via administration of the compound or agent to an animal model for AD or a human, and measuring the animal or human's responsiveness to the compound or agent. Compounds or agents to be assayed can include, without limitation, soluble CD40L, an antibody against CD40R that inhibits the CD40 pathway, an antibody against CD40L that inhibits the CD40 pathway, an antibody against CD40R that stimulates the CD40 pathway, a compound that blocks the CD40 pathway, a compound that interrupts CD40R with CD40L, a compound that stimulates the CD40 pathway, or a compound that stimulates CD40R interaction with CD40L. Animals can be examined for improvements in conditions described above or for improvements in β-amyloid deposition, soluble β-amyloid, inflammatory markers, microglial activation, astrocytic activation, neuronal apoptosis, neuronal necrosis, brain injury, tau phosphorylation, or tau paired helical filaments.

Also provided is a non-human transgenic animal model exhibiting one or more of the following: transgenic APP, overexpressed transgenic presenilin protein, overexpressed transgenic CD40 receptor, overexpressed transgenic CD40 ligand, and/or tau protein or mutants of the tau protein.

### BRIEF DESCRIPTION OF THE DRAWINGS

FIGS. 1a-n: Microgliosis and astrocytosis are reduced in Tg APP/CD40L-deficient mice by 16 months of age. Panels are representative 10× bright-field photomicrographs. FIGS. 1a-f: mouse brain sections stained with anti-CD11b antibody; left column represents sections from Tg APP_{sw} mice, and sections shown on the right were taken from Tg APP_{sw}/CD40L-deficient mice. Panels a and d represent cingulate cortices (CC); b and e, hippocampi (H); and c and f, enthorinal cortices (EC). FIGS. 1g-l: mouse brain sections stained with anti-GFAP antibody; left column represents sections from Tg APP_{sw} mice, and sections shown on the right were taken from Tg APP_{sw}/CD40L-deficient mice. Panels g and j represent CC; h and k, H; and i and l, EC. Scale bar denotes 100 μm (calculated for each panel). FIGS. 1m and n: percentage of microgliosis and percentage of astrocytosis, respectively. Percentages (mean±1 SEM) were calculated by quantitative image analysis, and percentage reduction for each brain region is indicated. The t-Test for independent samples revealed significant between-groups differences for each brain region examined in m and n (p<0.001 for each comparison).

FIGS. 2a-g: Congophilic amyloid deposits are markedly reduced in Tg APP_{sw}/CD40L-deficient mice by 16 months of age. Panels a-f are representative 10× bright-field photomicrographs of mouse brain sections stained with congo red. The left column represents sections from Tg APP_{sw} mice, and sections shown on the right were taken from Tg APP_{sw}/CD40L-deficient mice. Panels a and d represent cingulate cortices (CC); b and e, hippocampi (H); and c and f, enthorinal cortices (EC). Scale bar denotes 100 μm (calculated for each panel). Each of the left column panels show abundant congo red-positive amyloid deposits compared to the corresponding right panels. g, Congo red burden was calculated by quantitative image analysis (mean±1 SEM), and percentage reduction for each brain region is indicated. The t-Test for independent samples revealed significant between-groups differences for each brain region examined (p<0.001 for each comparison).

FIGS. 3a-h: Morphometric analysis of Aβ plaques in Tg APP_{sw}/CD40L-deficient mice. Panels a-f are representative 10× bright-field photomicrographs of mouse brain sections at 16 months of age stained with anti-Aβ antibody. The left column represents sections from Tg APP_{sw}/CD40L mice, and sections shown on the right were taken from Tg APP_{sw}/CD40L-deficient mice. Panels a and d represent cingulate cortices (CC); b and e, hippocampi (H); and c and f, enthorinal cortices (EC). Scale bar denotes 100 μm (calculated for each panel). Note the increased number of large diameter Aβ plaques in each of the left columns compared to corresponding right columns. Quantitative morphometric analysis results (mean plaque subtype per mouse±1 SEM), are displayed for g, the neocortex and h, the hippocampus, and percentage reduction of plaques in Tg APP_{sw}/CD40L-deficient-mice versus Tg APP_{sw}/CD40L mice is indicated. For g and h, t-Test for independent samples revealed significantly fewer large (greater than 50 μm) and medium-sized (between 25 and 50 μm) Aβ plaques in Tg APP_{sw}/CD40L-deficient mice compared to Tg APP_{sw}/CD40L mice (p<0.001 for each comparison).

FIGS. 4a-g: Reduced thioflavin S plaques in PSAPP mice treated with anti-CD40L antibody. Panels are 20× bright-field photomicrographs taken from 8-month old PSAPP mice that received anti-CD40L antibody or isotype-matched control IgG antibody. Figs. a-f: mouse brain sections stained with thioflavin S; left column shows sections from isotype-matched IgG-treated mice, and sections shown in the right column were taken from anti-CD40L antibody-treated mice. Panels a and d represent cingulate cortices (CC); b and e, hippocampi (H); and c and f, enthorinal cortices (EC). Fig. g: percentages of thioflavin-S-staining β-amyloid plaques (mean±1 SEM) were quantified by image analysis, and percentage reduction for each brain region is indicated. The t-Test for independent samples revealed significant between-groups differences for each brain region examined in g (p<0.001 for each comparison).

FIGS. 5a-e: CD40L modulates APP processing in vivo and in vitro. Brain homogenates were prepared from 12-month-old Tg APP_{sw}/CD40L-deficient, control IgG-treated PSAPP, and anti-CD40L antibody-treated PSAPP animals. Representative lanes are shown from each mouse group. FIG. 5a: Western immunoblot by antibody 369 against the cytoplasmic tail of APP reveals holo APP, and two bands corresponding to C99 (β-CTF) and C83 (α-CTF) as indicated (top panel). Antibody BAM-10 reveals Aβ species (lower panel). Figs. b and c: densitometry shows the ratio of C99 to C83, with n=5 for each mouse group. The t-Test for independent samples revealed significant differences for each comparison (p<0.001). Cell lysates and conditioned media were prepared from N2a cells overexpressing human APP and treated with 2 μg/mL of heat-inactivated CD40L (control) or CD40L protein (CD40 ligation) at the time points indicated. Fig. d: C-terminal fragments of APP were analyzed in cell lysates by Western immunoblot using antibody 369. Fig. e: Aβ₁₋₄₀ and Aβ₁₋₄₂ peptides were analyzed in human APP-overexpressing N2a cells by ELISA. Data are represented as percentage of Aβ peptide secreted after CD40 ligation relative to control protein treatment. ANOVA revealed a significant effect of incubation period on A-β₁₋₄₀ and A-β₁₋₄₂ (p<0.01). Data shown are representative of three independent experiments.

FIGS. 6a-e: Phospho-tau in situ by antibody pS199. 40× photomicrographs. Figs. a and b were taken from 16-month-old Tg APP_{sw} mice (n=4) and Figs. c and d are from age-matched TgAPP_{sw}/CD40L-deficient mice (n=5). Figs. a and c are from the neocortex and Figs. b and d are from the hippocampus. (*) indicates A [symbol] plaques. Quantitative analysis of pooled data is shown in Fig. e.

FIGS. 7a-e: Phospho-tau in situ by antibody pS202. 40× photomicrographs. Figs. a and b were taken from 16-month-old Tg APP_{sw} mice (n=4) and Figs. c and d are from age-matched Tg APP_{sw}/CD40L-deficient mice (n=5). Figs. a and c are from the neocortex and Figs. b and d are from the hippocampus. (*) indicates A [symbol] plaques. Quantitative analysis of pooled data is shown in Fig. e.

FIGS. 8a-d: β-amyloid deposits are markedly reduced in 8-month-old PSAPP mice treated with anti-CD40L antibody. Fig. a: mouse brain sections were stained with anti-Aβ antibody (4G8); left column shows sections from control IgG-treated mice, and sections shown in the right column were taken from anti-CD40L antibody-treated mice, as indicated. Top panels show cingulate cortices (CC); middle panels, hippocampi (H); and bottom panels, enthorinal cortices (EC), as indicated. Fig. b: percentages of 4G8-positive β-amyloid plaques (mean±1 SEM) were calculated by quantitative image analysis, and percentage reduction for each brain region is indicated. Fig. c: mouse brain sections from the indicated brain regions were stained with thioflavin S; left column shows sections from control IgG-treated mice, and sections shown in the right column were taken from anti-CD40L antibody-treated mice. Fig. d: percentages of thioflavin S plaques (mean±1 SEM) were calculated by quantitative image analysis, and percentage reduction for each brain region is indicated. t-Test for independent samples revealed significant between-groups differences for each brain region examined in b and d (p<0.001 for each comparison).

FIGS. 9a-f: CD40L modulates APP processing in vivo and in vitro. Fig. a: Brain homogenates were prepared from 12-month-old TgAPP_{sw}/CD40L-deficient, control IgG-treated PSAPP, and anti-CD40L antibody-treated PSAPP animals. Representative lanes are shown from each mouse group. Western immunoblot by antibody 369 against the cytoplasmic tail of APP revealed holo APP, and two bands corresponding to C99 (α-CTF) and C83 (β-CTF). Figs. b and c: Densitometry shows the ratio of C99 to C83, with n=5 for each mouse group. The t-Test for independent samples revealed significant differences for each comparison (p<0.001). Fig. d: Cell lysates were prepared from N2a cells overexpressing human wild-type APP-695 and treated with 2 μg/mL of heat-inactivated CD40L (control) or CD40L protein (CD40 ligation) for 24 hours. Fig. e: Densitometry shows the ratio of C99 to C83, with n=3 for each condition. One-way ANOVA revealed significant between-groups differences (p<0.001), and post-hoc comparison showed a significant difference between CD40L treatment and control (p<0.001). No significant difference was noted when comparing CD40L/anti-CD40L co-treatment to control, indicating complete blockade of the effect of CD40L. Fig. f: A-β₁₋₄₀ and A-β₁₋₄₂ peptides were analyzed in conditioned media from human wild-type APP-695 overexpressing N2a cells by ELISA (n=3 for each condition). Data are represented as percentage of Aβ peptide secreted 24 hours after CD40 ligation relative to heat-inactivated CD40L treatment. When measuring A-β₁₋₄₀ and A-β₁₋₄₂, one-way ANOVA revealed significant between-groups differences (p<0.001), and post-hoc comparison showed a significant difference between CD40L treatment and the CD40L/anti-CD40L antibody co-treatment condition (p<0.001), and no significant difference was noted when comparing CD40L/anti-CD40L co-treatment to untreated control treatment, indicating complete blockade of Aβ secretion induced by CD40L. Figs. d and e: co-treatment with CD40L and control IgG antibody did not produce a significant difference from CD40L treatment (data not shown). Similar results were obtained with antibody 6687 or Chemicon polyclonal APP C-terminal antibody (data not shown).

### DESCRIPTION OF THE PREFERRED EMBODIMENTS

The present invention provides methods for treating neuronal inflammation, brain injury, brain trauma, tauopathies, or amyloidogenic diseases, comprising the administration of therapeutically effective amounts of a composition comprised of an agent and a carrier that interferes with the CD40L/CD40R signaling pathway to an individual afflicted with neuronal inflammation, brain injury, brain trauma, tauopathies, or an amyloidogenic disease. Where tauopathies are to be treated, agents can be administered that reduce the phosphorylation of the tau protein or mutants thereof.

The present invention also provides methods for causing a desired biological effect, comprising the administration of a composition comprised of an agent and a carrier which interferes with the CD40L/CD40R signaling pathway to an individual or system in amounts sufficient to cause the desired biological effect. The phrase “interferes with the CD40L/CD40R signaling pathway” can be construed as disrupting the binding or association of CD40L with its cognate receptor, CD40R, or interfering with the trimerization of CD40R. Alternatively, the phrase can be construed as disrupting the signaling pathway upstream or downstream of CD40L/CD40R binding.

In one embodiment of the invention, the agent can be an anti-CD40L antibody, examples of which include, without limitation, one or more species of monoclonal antibody, polyclonal antibody, or a combination of polyclonal and monoclonal antibodies, which can be administered in amounts sufficient to cause a desired biological effect.

A “desired biological effect” can include, without limitation, modulating or altering APP processing in an individual or system, altering the ratio of APP β-CTF to APP α-CTF in an individual or system, reducing the β-CTF to α-CTF ratio in an individual or system, reducing the amount of β-CTF in an individual or system, promoting brain-to-blood clearance of Aβ in an individual or system, increasing circulating levels (concentrations of Aβ in an individual or system, decreasing levels of Aβ=0 in the CNS in an individual or system, reducing β-secretase and/or γ-secretase activity in an individual or system, or any combination thereof.

The term “CD40R” is interchangeable with the more generic term “CD40”, both terms signifying the CD40 receptor. The phrase “interferes with the CD40L/CD40R signaling pathway” can be construed as disrupting the binding or association of CD40L with its cognate receptor, CD40R, or interfering with the trimerization of CD40R. Alternatively, the phrase can be construed as disrupting the signaling pathway upstream or downstream of CD40L/CD40R binding.

In particular, one embodiment of the present invention provides a method for identifying compounds that modulate the CD40L/CD40R signaling pathway, comprising contacting CNS cells expressing CD40R with CD40L and a compound and measuring a marker; contacting peripheral cells expressing CD40R with CD40L and the compound and measuring a marker; contacting CNS cells with a stimulator of the CD40L/CD40R signaling pathway and a compound and measuring a marker; contacting peripheral cells with a stimulator of the CD40L/CD40R signaling pathway and the compound and measuring a marker; contacting CNS cells with an inhibitor of the CD40L/CD40R signaling pathway and the compound and measuring a marker; contacting peripheral cells with an inhibitor of the CD40L/CD40R signaling pathway and the compound and measuring a marker; and comparing the markers to identify those compounds that modulate the CD40L/CD40R signaling pathway.

CNS cells are cells including, without limitation, neurons, glia, and associated cells of the cerebrospinal vasculature. Peripheral cells are cells that are not CNS cells. Various other cells, in addition to CNS cells and peripheral cells, can be used to determine the modulatory effect of test compounds according to the methods of the present invention. Examples of other such cells include, without limitation, cell lines derived from CNS cells, cell lines derived from peripheral cells, transgenic cells, transgenic cells derived from transgenic animals, or human cells or cell lines. Examples of transgenic animals include, without limitation, transgenic worms, transgenic flies, or transgenic rodents.

Markers that can be measured include, without limitation, the levels or amounts of one or more cytokines, such as tumor necrosis factor, interleukin 1, interleukin 6, interleukin 12, interleukin 18, macrophage inflammatory protein, macrophage chemoattractant protein, granulocyte-macrophage colony stimulating factor, macrophage colony stimulating factor, or combinations thereof Other markers that can be measured can include, without limitation, glutamate release, nitric oxide production, nitric oxide synthase, superoxide, superoxide dismutase, or combinations thereof. Still other markers that can be measured can include, without limitation, a major histocompatability complex molecule, CD45, CD11b, integrins, a cell surface molecule, or combinations thereof. Further, markers that can be measured according to the methods of the present invention include, without limitation, the levels or amounts of Aβ, β-APP, a fragment of β-APP, a fragment of Aβ, or combinations thereof.

The types of compounds to be tested to determine their modulatory activity of the CD40L/CD40R signaling pathway according to the methods of the present invention include, without limitation, agonistic antibodies to CD40R and/or CD40L, antagonistic antibodies to CD40R and/or CD40L, compounds which bind to CD40L or decrease trimerization of CD40R, compounds which bind to CD40R or decrease trimerization of CD40R, or compounds which modulate the CD40L/CD40R signaling pathway upstream or downstream of CD40L/CD40R interaction.

Another embodiment of the present invention provides a method for identifying compounds that reduce, ameliorate, or modulate signs and/or symptoms associated with neuronal inflammation, brain injury, brain trauma, tauopathies, or amyloidogenic diseases, comprising administering a compound that modulates the CD40L/CD40R signaling pathway to an animal model and measuring or observing the reduction, amelioration, or modulation of the symptoms of the above-described afflictions.

Examples of the reduction, amelioration, or modulation of signs and/or symptoms associated with the above-described amyloidogenic diseases include, without limitation, reductions in the size and/or number of amyloid plaques, reduction in β-amyloid burden, reduction in soluble Aβ levels, reduction in total Aβ levels, reduction of congophilic β-amyloid deposits, reduction of reactive gliosis, microgliosis, astrocytosis, and combinations thereof.

A further embodiment of the present invention provides a method for treating neuronal inflammation, brain injury, brain trauma, tauopathies, or amyloidogenic diseases, comprised of administration to an individual therapeutically effective amounts of a composition containing an agent and a carrier which interferes with the CD40L/CD40R signaling pathway or the phosphorylation of tau protein.

Examples of compounds, agents or compositions that can be identified as reducing, ameliorating, or modulating signs and/or symptoms associated with neuronal inflammation, brain injury, brain trauma, tauopathies, or amyloidogenic diseases, and thus can be used to treat such afflictions include, without limitation, CD40L, soluble CD40L, immunogenic CD40L, CD40L variants (CD40LV), antibodies that bind to CD40L and block its interaction with CD40R, antibodies that bind to CD40R and block ligand binding to the receptor, soluble CD40LV that bind to CD40R and fails to activate the receptor, interfering RNA or antisense RNA to CD40R or CD40L, or combinations thereof.

Examples of amyloidogenic diseases include, without limitation, Alzheimer's disease, scrapie, transmissible spongiform encepalopathies, hereditary cerebral hemorrhage with amyloidosis Icelandic-type, hereditary cerebral hemorrhage with amyloidosis Dutch-type, familial Mediterranean fever, familial amyloid nephropathy with urticaria and deafniess (Muckle-Wells syndrome), myeloma or macroglobulinemia-associated idiopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes Type II, and insulinoma.

Examples of tauopathies include, without limitation, Alzheimer's disease, frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amyotrophy complex, Pick's disease, or Pick's disease-like dementia.

Yet another embodiment of the present invention provides a method for causing a desired biological effect, comprised of the administration of a composition containing an agent and a carrier which interferes with the CD40L/CD40R signaling pathway to an individual or system in amounts sufficient to cause the desired biological effect. The phrase “interferes with the CD40L/CD40R signaling pathway” can be construed as disrupting the binding or association of CD40L with its cognate receptor, CD40R, or interfering with the trimerization of CD40R. Alternatively, the phrase can be construed as disrupting the signaling pathway upstream or downstream of the CD40L/CD40R binding.

Examples of desired biological effects include, without limitation, modulating or altering APP in an individual or system, altering the ratio of APP β-C-terminal fragments, (β-CTF) to APP α-C-terminal fragments (α-CTF) in an individual or system, reducing the β-CTF to α-CTF ratio in an individual or system, reducing the amount of β-CTF in an individual or system, promoting brain-to-blood clearance of Aβ in an individual or system, increasing circulating levels (concentrations) of Aβ in an individual or system, decreasing levels of Aβ in the CNS in an individual or system, reducing β-secretase and/or γ-secretase activity in an individual or system, or any combination thereof.

The present invention also provides for the administration of anti-CD40 or anti-CD40L antibody, as an agent, in amounts sufficient to cause a desired biological effect in an individual or system. Anti-CD40 or anti-CD40L antibody compositions can include, without limitation, one or more species of monoclonal anti-CD40 or anti-CD40L antibodies, polyclonal antibodies to CD40 or CD40L, or a combination thereof.

Accordingly, the present invention provides methods of modulating or altering APP processing by administering an effective amount of a composition comprised of an agent and a carrier which interferes with the CD40L/CD40R signaling pathway to an individual or system. In one embodiment, APP processing is altered via the administration of anti-CD40R antibody to the system in amounts sufficient to alter the processing of APP. In another embodiment, APP processing is altered via the administration of anti-CD40L antibody to the system in amounts sufficient to alter the processing of APP.

Thus, the present invention can provide methods of altering the ratio of APP β-CTF to APP α-CTF by administering a composition comprised of an agent and a carrier that interferes with the CD40L/CD40R signaling pathway to a system or individual in amounts sufficient to alter the β-CTF to α-CTF ratio. In one embodiment, the β-CTF to α-CTF ratio is altered via the administration of anti-CD40R antibody to the system in amounts sufficient to alter the β-CTF to α-CTF ratio. In another embodiment, the β-CTF to α-CTF ratio is altered via the administration of anti-CD40L antibody to the system in amounts sufficient to alter the β-CTF to α-CTF ratio.

Also included in the scope of the invention are methods for reducing the amount of β-CTF in an individual or system by administering a composition comprised of an agent and a carrier which interferes with the CD40L/CD40R signaling pathway to a system or individual in amounts sufficient to reduce the amounts of β-CTF in an individual or system. In one embodiment, the amount of β-CTF in an individual or system is reduced via the administration of anti-CD40R antibody to the system in amounts sufficient to alter the β-CTF to α-CTF ratio. In another embodiment, the amount of β-CTF in an individual or system is reduced via the administration of anti-CD40L antibody to the system in amounts sufficient to alter the β-CTF to α-CTF ratio.

The present invention also provides methods for reducing β-secretase and/or γ-secretase activity in an individual or system by administering a composition comprised of an agent and a carrier that interferes with the CD40L/CD40R signaling pathway to a system or individual in amounts sufficient to reduce β-secretaase and/or γ-secretase activity in an individual or system. In one embodiment, the reduction of β-secretase and/or γ-secretase activity can be mediated via the administration of anti-CD40R antibody to the system in amounts sufficient to reduce β-secretase and/or γ-secretase activity. In another embodiment, the reduction of β-secretase and/or γ-secretase activity can be mediated via the administration of anti-CD40L antibody to the system in amounts sufficient to reduce β-secretase and/or γ-secretase activity.

Another embodiment of the present invention provides methods of promoting brain-to-blood clearance of Aβ in an individual or system by administering a composition comprised of an agent or carrier that interferes with the CD40L/CD40R signaling pathway to an individual or system in amounts sufficient to cause brain-to-blood clearance of Aβ in an individual or system.

The present invention also provides methods of increasing circulating levels, or concentrations, of Aβ in an individual or system by administering a composition comprised of an agent or carrier that interferes with the CD40L/CD40R signaling pathway to an individual or system in amounts sufficient to increase circulating levels, or concentrations, of Aβ in an individual or system.

CD40L refers to native, recombinant or synthetic forms of the molecule. Native, recombinant, or synthetic forms of CD40L (termed CD40L variants, or CD40LV) can contain amino acid substitutions, additions, or deletions that do not affect the ability of the ligand to bind to CD40R but, unlike the native CD40L (i.e., CD40L having the naturally occurring amino acid sequence and the ability to activate CD40R), such binding does not activate CD40R. In certain embodiments, CD40LV can bind to CD40R and, through competitive inhibition, block the binding of native CD40L to CD40R. Variants of CD40L (CD40LV) also can include, without limitation, isoforms of the CD40 ligand or fragments thereof that contain the binding site for CD40L, and thus are capable of binding to CD40R, but do not stimulate the CD40L/CD40R signaling pathway. The phrases “therapeutically effective amounts,” “amounts sufficient to,” or “effective amounts” are to be construed as an amount of a composition that confers an improvement in the condition of an individual treated according to the methods taught herein or amounts of a composition conferring the effect recited in the methodology (e.g., decreasing secretase cleavage activity or altering APP processing). Non-limiting examples of such improvements for an individual include improvements in quality of life and/or memory, reductions in the size and/or number of amyloid plaques, reduction in β-amyloid burden, reduction in congophilic β-amyloid deposits, reduction in reactive gliosis, microgliosis, and/or astrocytosis, an improvement in the symptoms with which an individual presents to a medical practitioner (i.e., reductions in the severity of symptoms with which the individual presents), or reduction of other β-amyloid-associated pathologies. The term “system” can be construed to include in vitro and/or in vivo systems. Non-limiting subsets of the term “system(s)” include “in vitro system(s)” and “in vivo system(s).”

An “agent that interferes with the interaction of CD40L and CD40R” includes, without limitation, soluble CD40R, antibodies that bind to CD40L and block its interaction with CD40R, antibodies that bind to CD40R and block ligand binding to the receptor, soluble CD40LV that bind to CD40R and fail to activate the receptor, agents that reduce or inhibit the trimerization of CD40R, interfering RNA (dsRNA or RNAi) that suppresses or reduces the levels of CD40R expression, antisense RNA to CD40R (in amounts sufficient to suppress or reduce the levels of CD40R expression), RNAi that reduces the levels or amounts of Aβ protein that is expressed and that blocks or suppresses/reduces the ability of Aβ to induce CD40R expression, or antibodies that bind to Aβ and block or suppress/reduce its ability to induce CD40R expression. Antibodies that bind to CD40R can agonize or, preferably, antagonize the function of the receptor. In some embodiments, CD40L is rendered immunogenic according to methods known in the art and used to engender an immune response to native CD40L. Antibodies suitable for use in the present invention can be purchased from commercial sources or made according to methods known in the art.

Methods of making soluble CD40L are known in the art (see for example U.S. Pat. No. 5,962,406 which is hereby incorporated by reference in its entirety) as are methods of interfering with CD40L/CD40R interactions (see for example U.S. Pat. No. 6,264,951, also hereby incorporated by reference in its entirety). Likewise, methods of mutagenizing receptor ligands and analyzing the effects of such mutagenesis on receptor ligand interaction is well-known in the art and are described in the aforementioned U.S. patents.

Antisense technology also can be used to interfere with the CD40L/CD40R signaling pathway. For example, the transformation of a cell or organism with the reverse complement of a gene encoded by a polynucleotide exemplified herein can result in strand co-suppression and silencing or inhibition of a target gene, e.g., Aβ, CD40L, or CD40R.

Therapeutic protocols and methods of practicing antisense therapies for the modulation of CD40R are well-known to the skilled artisan (see for example, U.S. Pat. Nos. 6,197,584 and 6,194,150, each of which is hereby incorporated by reference in its entirety).

The ability to specifically inhibit gene function in a variety of organisms utilizing antisense RNA or dsRNA-mediated interference (RNAi or dsRNA) is well-known in the field of molecular biology (see for example C. P. Hunter, (1999) Current Biology, 9:R440-442; Hamilton et al., (1999) Science, 286:950-952; and S. W. Ding, (2000) Current Opinions in Biotechnology, 11:152-156, hereby incorporated by reference in their entireties). Interfering RNA, either double-stranded interfering RNA (dsRNAi or dsRNA) or RNA-mediated interference (RNAi), typically comprises a polynucleotide sequence identical or homologous to a target gene, or fragment of a gene, linked directly, or indirectly, to a polynucleotide sequence complementary to the sequence of the target gene or fragment thereof. The dsRNAi may comprise a polynucleotide linker sequence of sufficient length to allow for the two polynucleotide sequences to fold over and hybridize to each other, although a linker sequence is not necessary. The linker sequence is designed to separate the antisense and sense strands of RNAi significantly enough to limit the effects of steric hindrance and allow for the formation of dsRNAi molecules and should not hybridize with sequences within the hybridizing portions of the dsRNAi molecule. The specificity of this gene silencing mechanism appears to be extremely high, blocking expression only of targeted genes, while leaving other genes unaffected. Accordingly, one method for treating amyloidogenic diseases according to the present invention includes the use of materials and methods utilizing either dsRNA or RNAi comprised of polynucleotide sequences identical or homologous to CD40L and/or CD40R. The terms “dsRNAi,” “RNAi,” and “siRNA” are used interchangeably herein unless otherwise noted.

RNA containing a nucleotide sequence identical to a fragment of the target gene is preferred for inhibition; however, RNA sequences with insertions, deletions, and point mutations relative to the target sequence can also be used for inhibition. Sequence identity may be optimized by sequence comparison and alignment algorithms known in the art (see Gribskov and Devereux, Sequence Analysis Primer, Stockton Press, 1991, and references cited therein) and then calculating the percent difference between the nucleotide sequences by, for example, the Smith-Waterman algorithm as implemented in the BESTFIT software program using default parameters (e.g., University of Wisconsin Genetic Computing Group). Alternatively, the duplex region of the RNA may be defined functionally as a nucleotide sequence that is capable of hybridizing with a fragment of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand(s); the promoters may be known inducible promoters, such as baculovirus. Inhibition may be targeted by specific transcription in an organ, tissue, or cell type. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see for example, WO 97/32016; U.S. Pat. Nos. 5,593,874; 5,698,425; 5,712,135; 5,789,214; and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

Preferably, and most conveniently, dsRNAi can be targeted to an entire polynucleotide sequence, such as CD40R, CD40L, or Aβ. Preferred RNAi molecules of the present invention are highly homologous or identical to the polynucleotides encoding CD40R, CD40L or Aβ. The homology may be greater than 70%, preferably greater than 80%, more preferably greater than 90% and is most preferably greater than 95%.

Fragments of genes also can be utilized for targeted suppression of gene expression. These fragments are typically in the approximate size range of about 20 consecutive nucleotides of a target sequence. Thus, targeted fragments are preferably at least about 16 consecutive nucleotides. In certain embodiments, the gene fragment targeted by the RNAi molecule is about 20-25 consecutive nucleotides in length. In a more preferred embodiment, the gene fragments are at least about 25 consecutive nucleotides in length. In an even more preferred embodiment, the gene fragments are at least 50 consecutive nucleotides in length. Various embodiments also allow for the joining of one or more gene fragments of at least about 15 nucleotides via linkers. Thus, RNAi molecules useful in the practice of the present invention can contain any number of gene fragments joined by linker sequences.

In yet other embodiments, the gene fragments can range from one nucleotide less than the full length gene (X_{CD40L}=n−1; X_{CD40R}=n−1; or X_{Aβ}=n−1, where X is a given whole number fragment length and n is the number of nucleotides in the full length CD40L, CD40R, or Aβ sequence). Nucleotide sequences for CD40R, CD40L, and Aβ are known in the art and can be obtained from patent publications, public databases containing nucleic acid sequences, or commercial vendors. This paragraph is also to be construed as providing written support for any fragment length ranging from 15 consecutive polynucleotides to one nucleotide less than the full length polynucleotide sequence of CD40L, CD40R, or Aβ; thus X_{CD40L}, X_{CD40R}, or X_{Aβ} can have a whole number value ranging from 15 consecutive nucleotides to one nucleotide less than the full length polynucleotide.

Accordingly, methods utilizing RNAi molecules in the practice of the present invention are not limited to those that are targeted to the full length polynucleotide or gene. Gene product can be inhibited with an RNAi molecule that is targeted to a portion or fragment of the exemplified polynucleotides; high homology (90-95%) or greater identity is also preferred, but not essential, for such applications.

In another aspect of the present invention, the dsRNA molecules of the invention may be introduced into cells with single stranded RNA molecules (ssRNA) which are sense or anti-sense RNA derived from the nucleotide sequences disclosed herein. Methods of introducing ssRNA and dsRNA molecules into cells are well-known to the skilled artisan and include transcription of plasmids, vectors, or genetic constructs encoding the ssRNA or dsRNA molecules according to this aspect of the invention. Electroporation, biolistics, or other well-known methods of introducing nucleic acids into cells may also be used to introduce the ssRNA and dsRNA molecules of this invention into cells.

In another embodiment of the present invention, methods are provided for the treatment of internal organ diseases related to amyloid plaque formation, including plaques in the heart, liver, spleen, kidney, pancreas, brain, lungs and muscles, by administering therapeutically effective amounts of a composition comprised of an agent and a carrier which interferes with the CD40L/CD40R signaling pathway to an individual in need of such treatment.

In still another embodiment of the present invention, assays are provided for the identification of small molecules or other compounds capable of modulating CD40L/CD40R signaling pathways. The assays can be performed in vitro using non-transformed cells, immortalized cell lines, recombinant cell lines, transgenic cells, transgenic cell lines, or transgenic animal and cells/cell lines derived therefrom. Transgenic animals suitable for use in the present invention include, without limitation, transgenic worms, transgenic flies, or transgenic mice. For in vitro assays, cells and cell lines can be of human or other animal origin. In particular, the assays can be used to examine the effects of small molecules or other compounds on neuronal inflammation, brain injury, tauopathies, or an amyloidogenic disease. In such assays, the small molecules or other compounds can be tested for the ability to elicit an improvement in the condition of an individual to be treated according to the methods taught herein. Thus, for example, cells can be examined for decreased inflammation or other suitable changes in markers that are well-known in the art. Additionally, the present invention provides in vivo methods for identifying small molecules or other compounds capable of modulating CD40L/CD40R signaling pathways via the administration of such compounds to individuals or animals (e.g., human volunteers or murine models) and examining the individuals or animals for an improvement in the condition of the individual or animal treated according to the methods taught herein.

The present invention also provides therapeutic compounds or small molecules and compositions comprised of a carrier and the therapeutic compounds or small molecules. In certain embodiments, the carrier is a pharmaceutically acceptable carrier or diluent.

Compositions containing therapeutic compounds and/or small molecules can be administered to a patient via various routes including parenterally, orally or intraperitoneally. Parenteral administration includes the following routes: intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, or nasal inhalation via insufflation or nebulization.

Compounds or small molecules that are orally administered can be enclosed in hard or soft shell gelatin capsules, or compressed into tablets. Active compounds or small molecules also can be incorporated with an excipient and used in the form of ingestible tablets, buccal tablets, troches, capsules, sachets, lozenges, elixirs, suspensions, syrups, wafers, and the like. The pharmaceutical composition containing the active compounds can be in the form of a powder or granule, a solution or suspension in an aqueous liquid or non-aqueous liquid, or in an oil-in-water or water-in-oil emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain the active compound, sucrose as a sweetening agent, methyl and propylparabens as preservatives, a dye and flavoring. Any material used in preparing any dosage unit form should be pharmaceutically pure and substantially non-toxic. Additionally, the active compound can be incorporated into sustained-release preparations and formulations.

The active compounds can be administered to the CNS, parenterally or intraperitoneally. Solutions of the compound as a free base or a pharmaceutically acceptable salt can be prepared in water mixed with a suitable surfactant, such as hydroxypropylcellulose. Dispersions also can be prepared glycerol, liquid polyethylene glycols, and mixtures thereof, and in oils. Under ordinary conditions of storage and use, these preparations can contain a preservative and/or antioxidants to prevent the growth of microorganisms or chemical degeneration.

The pharmaceutical forms suitable for injectable use include, without limitation, sterile aqueous solutions or dispersions and sterile powders for the extemporaneous preparation of sterile injectable solutions or dispersions. In all cases, the form must be sterile and must be fluid to the extent that easy syringability exists. It can be stable under the conditions of manufacture and storage and must be preserved against the contaminating action of microorganisms, such as bacteria and fungi. The carrier can be a solvent or dispersion medium which contains, for example, and without limitation, water, ethanol, polyol (such as glycerol, propylene glycol, and liquid polyethylene glycol), suitable mixtures thereof, or vegetable oils. The proper fluidity can be maintained, for example, by the use of a coating, such as lecithin, by the maintenance of the required particle size (in the case of a dispersion) and by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars or sodium chloride.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and any of the other ingredients from those enumerated above. In the case of sterile powders for the preparation of sterile injectable solutions, the preferred methods of preparation are vacuum drying and freeze drying.

Pharmaceutical compositions which are suitable for administration to the nose or buccal cavity include, without limitation, self-propelling and spray formulations, such as aerosol, atomizers and nebulizers.

The therapeutic compounds of the present invention can be administered to a mammal alone or in combination with pharmaceutically acceptable carriers or as pharmaceutically acceptable salts, the proportion of which is determined by the solubility and chemical nature of the compound, chosen route of administration, and standard pharmaceutical practice.

The compositions also can contain other therapeutically active compounds which are usually applied in the treatment of the diseases and disorders discussed herein. Treatments using the present compounds and other therapeutically active compounds can be ultaneous or in intervals.

### EXAMPLE 1

### Genetic Disruption of CD40R/CD40L in Mammals

Genetic disruption of CD40L in Tg APP_{sw} mice results in reduced activation of microglia and astrocytes. These changes are concomitant with reduced Aβ pathology, with the most notable diminution in mature congophillic β-amyloid plaques at 16 months of age by 77-85%. Correspondingly, large (greater than 50 μm) and medium sized (between 25 and 50 μm) Aβ plaques are reduced by approximately the same amount in these animals. These data indicate that CD40R/CD40L signaling is important for the development of Aβ pathology.

Genetic disruption of CD40L in Tg APP_{sw} mice also results in reduced soluble and deposited Aβ levels, with up to 85% diminution, or more, of mature congophillic β-amyloid plaques. Correspondingly, large (greater than 50 μm) and medium-sized (between 25 and 50 μm) β-amyloid plaques are diminished by a comparable magnitude in these animals. These changes are concomitant with reduced brain inflammation as measured by reactive astrocytes and microglia. Disruption of the CD40R/CD40L signaling also reduces the incidence of Aβ pathology development and the late-stage maturation of β-amyloid plaques.

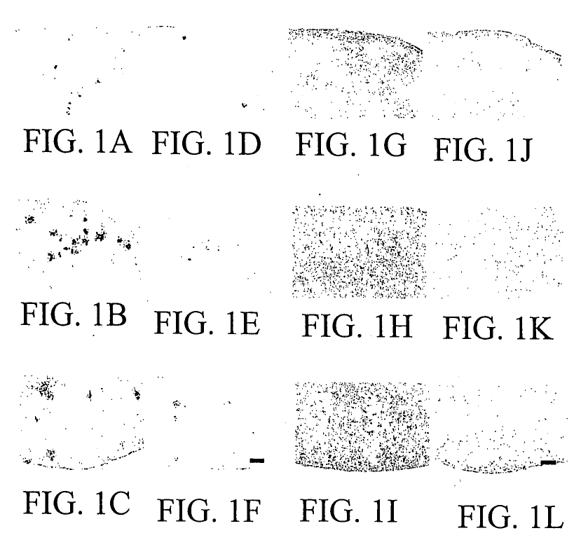

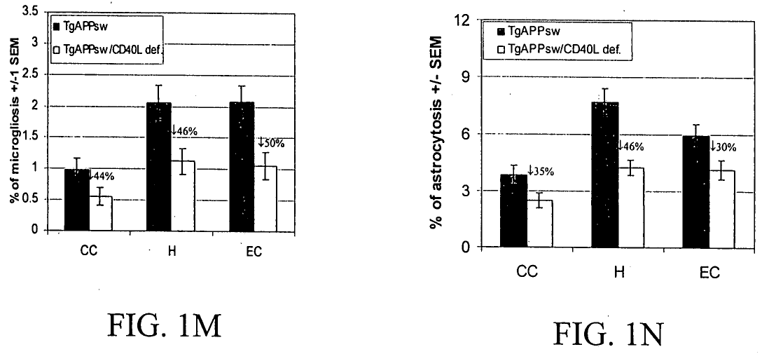

Tg APP_{sw} mice manifest prominent astrocytosis and microgliosis and develop amyloid deposits comparable to human senile plaques by 16 months of age (Irazarry et al., “APP_{sw} transgenic mice develop age-related A beta deposits and neurophil abnormalities, but no neural loss in CA1,” J. Neuropathol. Exp. Neurol. (1977) 56:965-73). To evaluate whether CD40L deficiency might oppose gliosis in Tg APP_{sw} mice, we performed immunohistochemistry for detection of CD11b (a marker of activated microglia and glial fibrillary acidic protein (GFAP, increased in activated astrocytes). As shown in FIGS. 1a-f, activated microglia appeared to be reduced in Tg APP_{sw}/CD40L def. mice compared to Tg APP_{sw} mice in each of the three brain regions examined (cingulate cortex, hippocampus, and enthorhinal cortex). Quantitative image analysis revealed significant differences for each brain region, showing between 44 and 50% reduction in activated microglia (FIG. 1m). Examination of GFAP-positive astrocytes showed a similar pattern of results, with diminished astrocytic activation ranging from 30 to 46% (FIGS. 1g-l, n). Additionally, measurement of brain TNF-α (an activated microglial marker that we have shown is secreted after Aβ and CD40L challenge (Tan et al., “Microglial activation resulting from CD40R/CD40L interaction after beta-amyloid stimulation,” Science (1999) 286:2352-55) protein levels by Western immunoblot revealed a statistically significant (p<0.001) 64% reduction in Tg APP_{sw}/CD40L def. mice compared to Tg APP_{sw} mice (mean TNF-α to actin ratio±1 SEM:Tg APP_{sw} mice, 0.247±0.02; control littermates, 0.13±0.01; Tg APP_{sw}/CD40L def. mice, 0.09±0.01; CD40L def. mice, 0.09±0.02), providing further evidence of reduced inflammation in Tg APP_{sw}/CD40L def. mouse brains.

In order to determine if the observed reduction in brain inflammation was associated with diminished Aβ pathology Tg APP_{sw}/CD40L def. mice, we evaluated the latter by four strategies: antibody immunoreactivity (conventional “Aβ burden” analysis), Aβ sandwich enzyme-linked immunoabsorbance assay (ELISA), congo red staining, and Aβ plaque morphometric analysis. While 12-month old Tg APP_{sw} mice had minimal Aβ plaque loads (≦2 plaques per section examined), Aβ plaques were not detectable in age-matched Tg APP_{sw}/CD40L def. mice (data not shown). In 16-month-old mice, up to 51% diminution of Aβ burden was evident in Tg APP_{sw}/CD40L def. mice for the brain regions examined, differences that were statistically significant (mean %±1 SEM; 41% reduction in cingulate cortex: Tg APP_{sw}, 1.74±0.22; Tg APP_{sw}/CD40L def. 1.02±0.10, p<0.05; 46% reduction in entorhinal cortex: Tg APP_{sw}, 1.12±0.16; Tg APP_{sw}/CD40L def., 0.60±0.06 p<0.001; 51% reduction in hippocampus: Tg APP_{sw} 0.79±0.08 Tg APP_{sw}/CD40L def., 39±0.08, p<0.001). Total Aβ ELISA analysis of these animals produced consistent results [mean Aβ (ng/wet g of brain)±1 SEM of Tg APP_{sw} mice vs. Tg APP_{sw}/CD40L def. mice; 45% reduction in Aβ₁₋₄₀: 569.01±15.80 vs. 315.04±62.29; 24% reduction in Aβ₁₋₄₂: 469.64±35.20 vs. 355.71±18.85; 35% reduction in total Aβ: 1038.66±21.83 vs. 670.75±81.14]. Analysis of total APP by Western immunoblot did not reveal a significant difference between these mice (mean APP to actin ratio±1 SEM; Tg APP_{sw} mice, 1.16±0.06; Tg APP_{sw}/CD40L def. mice, 1.15±0.04), suggesting that the observed differences on reduction of Aβ in Tg APP_{sw} mice deficient for CD40L are not due to down-regulation of APP production.

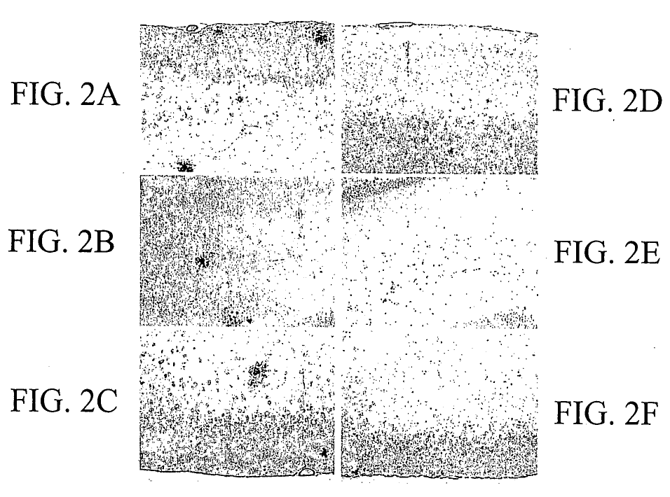

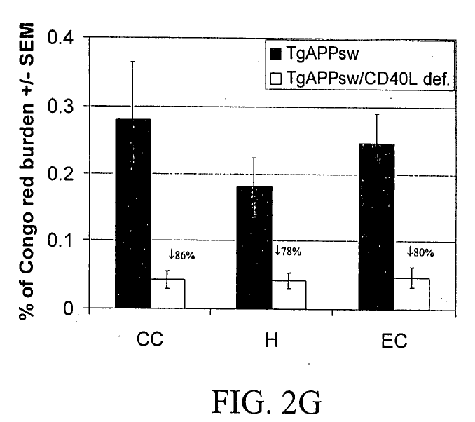

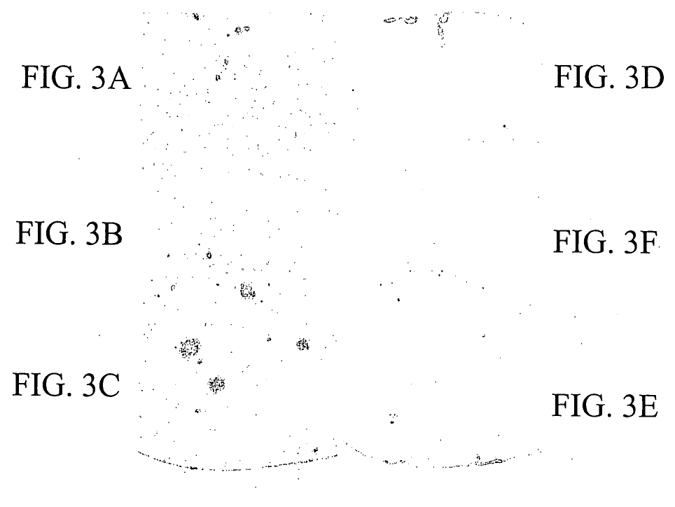

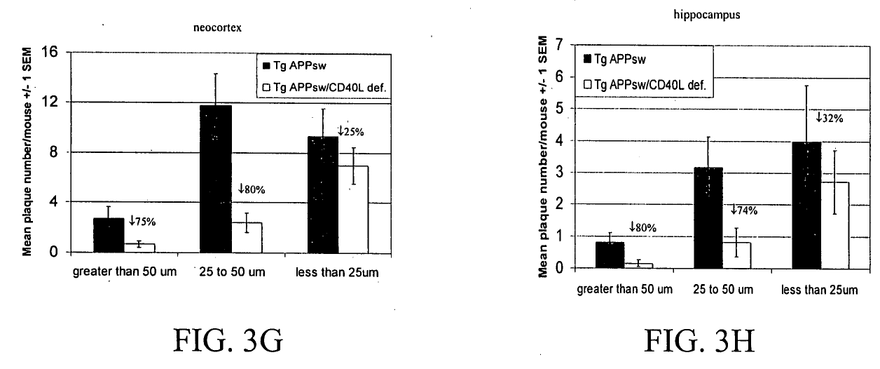

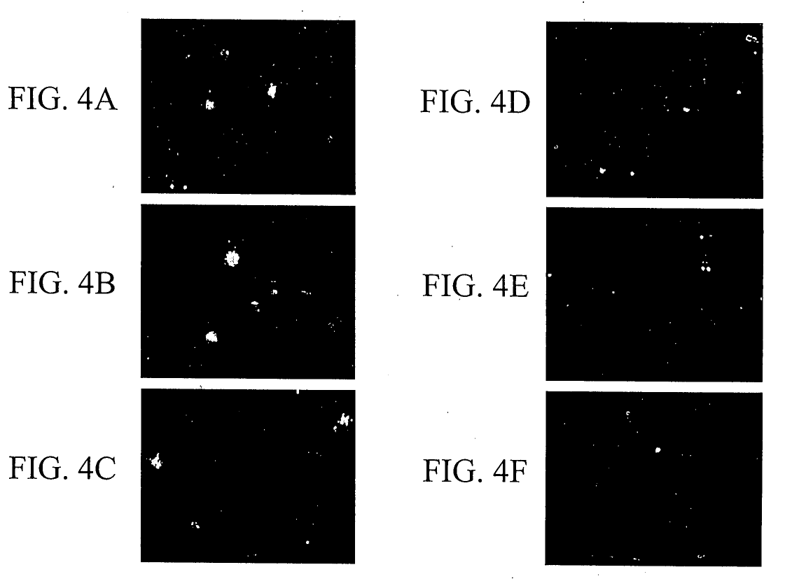

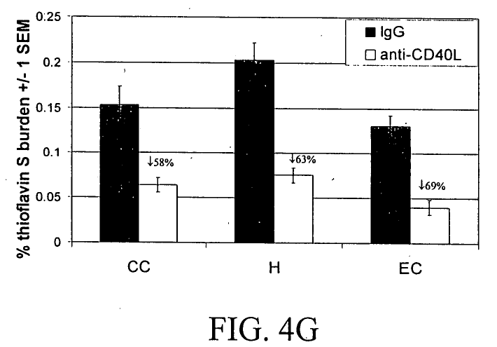

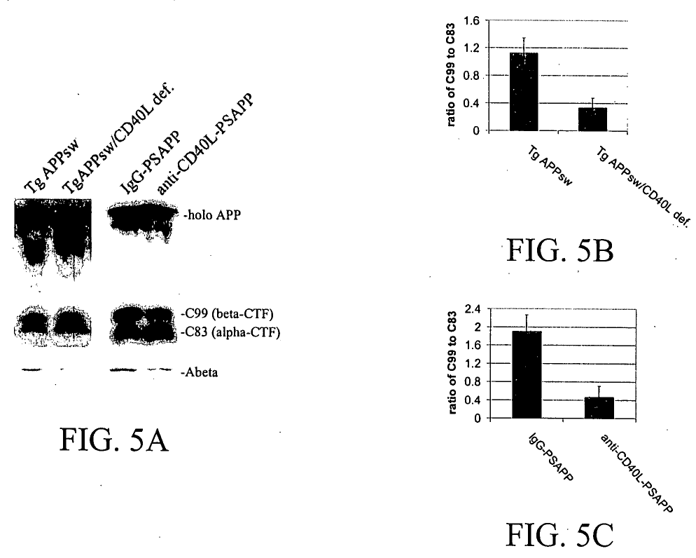

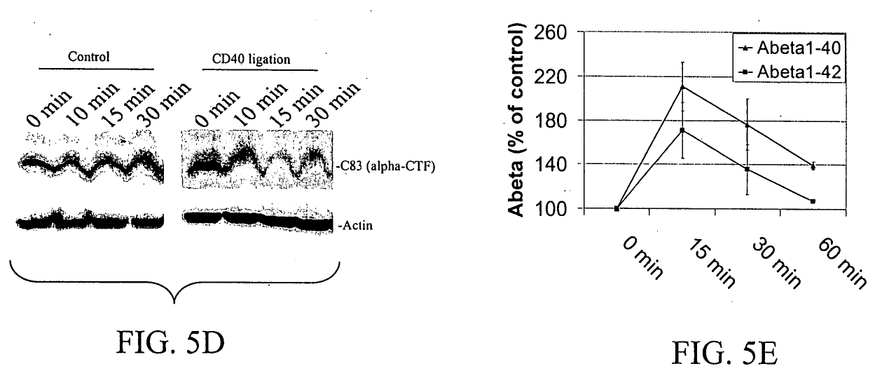

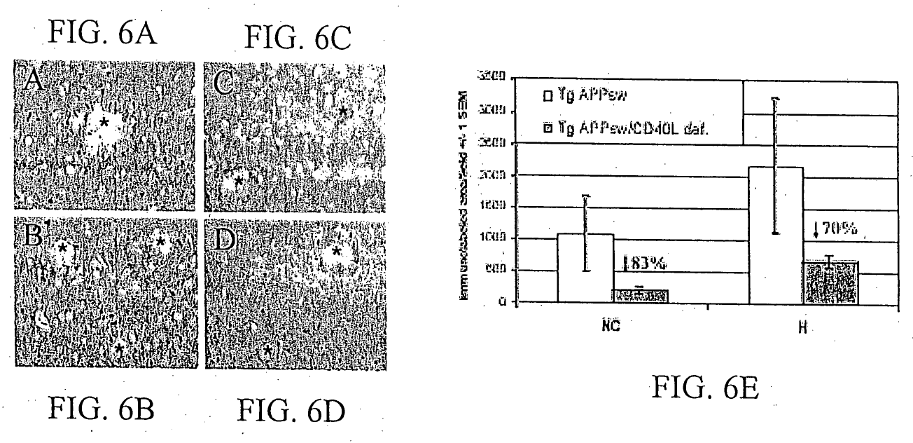

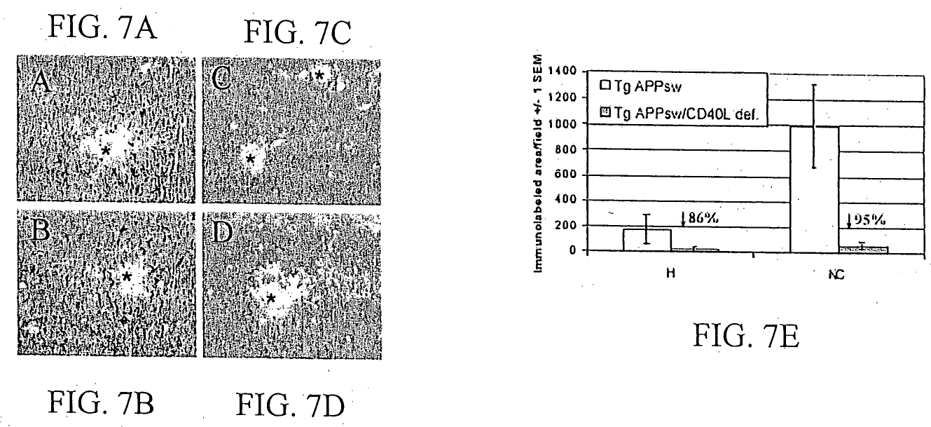

When taken together, our data indicate that blockade of the Aβ-mediated brain inflammatory response by opposing CD40 signaling provides a novel therapeutic target in AD. Additionally, these data support the hypothesis that CD40-mediated brain inflammation is detrimental by promoting Aβ pathology, most likely by affecting microglial activation. The effects reported here on CD40-mediated microgliosis, astrocytosis, and Aβ deposition could also be interpreted within the framework of the CD40R/-CD40L interaction as a key regulator of the peripheral immune response. As reduction in Aβ load in Tg APP_{sw}/CD40L def. mice was not complete, we hypothesized that interrupting CD40R-CD40L signaling might specifically mitigate formation of the mature, congophillic subset of Aβ plaques. Strikingly, data show between 78 and 86% reduction in congophilic plaques in Tg APP_{sw}/CD40L def. mice (FIG. 2). Morphometric analysis of anti-Aβ antibody immunoractive Aβ plaques at this age corroborates these data, showing a similar magnitude of reduction in large (>59 μm) and medium-sized (between 25 and 50 μm) Aβ plaque subsets in the neocortices and hippocampi of Tg APP_{sw}/CD40L def mice (FIG. 3). Similar to a previous finding implicating CD40L as required for the progression of atherosclerotic plaques (Lutgens et al., “Requirement for CD154 in the progression of atherosclerosis,” Nat. Med. (1999) 5:1313-16), the data presented here particularly support a role of the CD40R/CD40L interaction in the late stage maturation of Aβ plaques.

Immunohistochemistry. Standard methods known in the art and not specifically described are generally followed as in Stites et al. (eds.), Basic and Clinical Immunology (8^{th} Edition), Appleton & Lange, Norwalk, Conn. (1994) and Johnstone & Thorpe, Immunochemistry in Practice, Blackwell Scientific Publications, Oxford, 1982. General methods

in molecular biology: Standard molecular biology techniques known in the art and not specifically described are generally followed as in Sambrook et al, Molecular Cloning: A Laboratory Manual, Cold Springs Harbor Laboratory, New York (1989), 1992), and in Ausubel et al., Current Protocols in Molecular Biology, John Wiley and Sons, Baltimore, Md. (1989).

Mice. CD40L deficient mice are the C57BL/6 background and were constructed as previously described (Xu et al., “Mice deficient for the CD40 ligand,” Immunity (1994) 1:423-31). Tg APP_{sw} mice are the 2576 line crossed with C57B6/SJL as previously described (Hsiao et al., “Age-related CNS disorder and early death in transgenic FVB/N mice overexpressing Alzheimer amyloid precursor proteins,” Neuron (1995) 15:1203-18. We crossed CD40L deficient mice with Tg APP_{sw} transgenic mice and characterized first and second filial offspring by polymerase chain reaction-based genotyping for the mutant APP construct (to examine Tg APP_{sw} status) and neomycin selection vector (to type for CD40L deficiency), followed by Western blot for brain APP and splenic CD40L protein respectively. The animals that we then studied at 12 and 16 months of age were Tg APP_{sw}/CD40L deficient (Tg APP_{sw}/CD40L def.; 12 months: 3 female, 16 months: 3 female/1 male), non-Tg APP_{sw}/CD40L deficient (CD40L def.; 12 months: 3 female, 16 months: 3 female/1 male), Tg APP_{sw}/CD40L wild-type (Tg APP_{sw}; 12 months: 3 female, 16 months: 2 female/1 male), and non-Tg APP_{sw}/CD40L wild-type control littermate mice (Control; 12 months: 3 female, 16 months: 2 female/1 male).

Mice were anesthetized with isofluorane and transcardinally perfused with ice-cold physiological saline containing heparin. Brains were rapidly dissected and quartered using a mouse brain slicer (Muromachi Kikai Co., Tokyo, Japan). The first and second anterior quarters were homogenized for Western blot analyses, and the third and fourth posterior quarters were used for microtome or cryostat sectioning. For microgliosis analysis, brains were quick-frozen at −80° C., and for Aβ immunohistochemistry, congo red staining, and astrocytosis, brains were immersed in 4% paraformaldehyde at 4° C. overnight, and routinely processed in paraffin. Five coronal sections from each brain (5 μm) thickness) were cut with a 150 μm interval for these analyses. Immunohistochemical staining was performed in accordance with the manufacturer's instruction using the VECTASTAIN® Elite ABC kit (Vector Laboratories, Burlingame, Calif.), except that, for CD11b staining, a biotinylated secondary mouse IgG absorbed anti-rat antibody was used in place of the biotinylated anti-rabbit antibody that was supplied with the kit. Congo red staining was performed according to standard practice using 10% (w/v) filtered congo red dye cleared with alkaline alcohol, and methyl green was used for counter-staining. The following antibodies were variously employed for immunohistochemical staining: rabbit anti-cow GFAP antibody (1:500; DAKO, Carpinteria, Calif.), rabbit anti-human amyloid-β antibody (1:100; Sigma, Hercules, Mo.) and rat anti-mouse CD11b antibody (1:200; CALTAG LABORATOIRES, Burlingame, Calif.). Images were acquired from an Olympus BX60 microscope with an attached CCD video camera system (Olympus, Tokyo, Japan), and video signal was routed into a Windows 98SE™ PC via an AG5 averaging flame grabber (Scion Corporation, Frederick, Md.) for quantitative analysis using Image-Pro software (Media Cybernetics, Md.). Images of five 5 μm sections (150 μm apart) through each anatomic region of interest (hippocampus or cortical areas) were captured and a threshold optical density was obtained that discriminated staining from background. Manual editing of each field was used to eliminate artifacts. For Aβ or congo red burden, astrocytosis and microgliosis analyses, data are reported as the percentage of immunolabeled area captured (positive pixels) divided by the full area captured (total pixels). for Aβ plaque morphometric analysis, diameters of Aβ plaques were calculated via quantitative image analysis and numbers of plaques falling into each diameter category were totaled. Each immunohistochemical analysis was performed by a single examiner (T.M. or T.T.) blinded to sample identities.

Mouse brains (Control, Tg APP_{sw}, CD40L def., and Tg APP_{sw}/CD40L def.) were isolated under sterile conditions on ice and placed in ice-cold lysis buffer) containing 20 mM Tris pH 7.5, 150 mM NaCl, 1 mM EDTA, 1 mM EGTA, 1% v/v Triton X-100, 2.5 mM sodium pyrophosphate, 1 mM β-glycerolphosphate, 1 mM Na3VO4, 1 μgmL leupeptin, and 1 mM PMSF). Brains were then sonicated on ice for approximately 3 min, let stand for 15 min. at 4° C., and centrifuged at 15,000 rpm for 15 min. Total Aβ species were detected by acid extraction of brain homogenates in 5M guanidine buffer (Johnson-Wood et al., “Amyloid precursor protein processing and A beta42 deposition in a transgenic mouse model of Alzheimer disease,” Proc. Natl. Acad. Sci. USA (1997) 94:1550-5), followed by a 1:10 dilution if lysis buffer, and Aβ1-40, Aβ1-42, and total Aβ (estimated by summing Aβ1-40, Aβ1-42 values) were quantified in these samples using the Aβ1-40, Aβ1-42 enzyme-linked immunosorbent assay (ELISA) kits (QCB, Hopkinton, Mass.), in accordance with the manufacturer's instructiobn, except that standards were diluted such that the final concentration included 0.5 M guanidine buffer. Total protein was quantified in brain homogenates using the Bio-Rad protein assay (Bio-Rad, Hercules, Calif.); thus, ELISA values are reported as ng of Aβ1-x/wet g of brain.

All data in this example were found to be normally distributed; therefore, in instances of single mean comparison, Levene's test for equality of variances followed by t-Test for independent samples were used to assess significance. In instances of multiple mean comparisons, analysis of variance (ANOVA) was employed, followed by post-hoc comparison using Bonferroni's method. For all analyses, alpha levels were set at 0.05 and analyses were performed using SPSS for Windows, release 10.0.5.

### EXAMPLE 2

### Exogenous Disruption of CD40L Function

Exogenous disruption of CD40L function was examined for the ability to produce a similar phenotypeas genetic ablation in a transgenic mouse model of accelerated cerebral amyloidosis. Animals were treated with anti-CD40L antibody and a comparable reduction of 4G8-positive and thioflavin S-staining β-amyloid plaques were observed. Attenuated Aβ/β-amyloid pathology in both of these scenarios is associated with modulation of APP processing towards the non-amyloidogenic pathway, as the potentially amyloidogenic β-C-terminal fragment (β-CTF) of the amyloid precursor protein (APP) is markedly reduced relative to the α-C-terminal fragment (α-CTF).

We sought to determine the impact of reducing CD40L availability on Aβ/β-amyloid pathology in a mouse model of AD that overproduces Aβ1-40 and Aβ1-42 and develops significant amyloid deposits by 16 months of age (Tg APPsw, line 2576) (Hsiao et al., “Correlative memory deficits, Abeta elevation, and amyloid plaques in transgenic mice,” Science (1996) 274:99-102). Thus, we crossed Tg APPsw mice with animals deficient in CD40L (Tg APP_{sw}/CD40L def.) (Tan et al., “Microglial activation resulting from CD40-CD40L interaction after beta-amyloid stimulation,” Science (1999) 286:2352-55).

In order to determine if genetic disruption of CD40L could produce dimished Aβ/β-amyloid pathology in Tg APP_{sw}/CD40L def. mice, we evaluated this pathology by four strategies: anti-Aβ antibody immunoreactivity (conventional “β-amyloid burden” analysis), Aβ sandwich enzyme-linked immunoabsorbance assay (ELISA), congo red staining, and β-amyloid plaque morphometric analysis. While 12-month old Tg APP_{sw} mice had minimal-amyloid plaque loads (≦2 plaques per section examined), β-amyloid plaques were not detectable in age-matched Tg APP_{sw}/CD40L def. mice. Sixteen (16)-month-old Tg APP_{sw} mice had typical β-amyloid load (Irizarry et al., “APPSw transgenic mice develop age-related A beta deposits and neuropil abnormalities, but no neuronal loss in CA1,” Neuropathol. Exp. Neurol. (1997) 56:965-73), up to 51% diminution of β-amyloid burden was evident in Tg APP_{sw}/CD40L def. compared to Tg APP_{sw} mice for the brain regions examined, differences that were statistically significant (mean %±1 SEM; 41$ reduction in cingulate cortex: Tg APP_{sw}, 1.74±0.22; Tg APP_{sw}/CD40L def., 102±0.10, p<0.05; 46% reduction in entorhinal cortex: Tg APP_{sw}, 1.12±0.16; Tg APP_{sw}/CD40L def., 60±0.06, p<0.001; 51% reduction in the hippocampus: Tg APP_{sw}, 79±0.08; Tg APP_{sw}/CD40L def., 0.39±0.08, p<0.001). Aβ ELISA analysis of these animals produced results consistent with the above findings [mean Aβ (ng/wet g of brain)±1 SEM of Tg APP_{sw} mice vs. Tg APP_{sw}/CD40L def. mice; 45% reduction in Aβ₁₋₄₀: 569.0±15.8 vs. 315.0±62.3; 24% reduction in Aβ₁₋₄₂: 469.6±35.2 vs. 355.7±18.9; 35% reduction in total Aβ: 1038.7±21.8 vs. 670.8±81.1, p<0.001 for each comparison]. Most notably, congophilic β-amyloid deposits were markedly reduced in Tg APP_{sw}/CD40L def. mice, as our data show a 78% (H) to 86% (CC) reduction compared to Tg APP_{sw} mice. In addition, morphometric analysis of anti-Aβ antibody immunoreactive β-amyloid plaques at this age showed a reduction in large (>50 μm) and medium-sized (between 25 and 50 μm) β-amyloid plaque subsets in their neocortices and hippocampi. Analysis of total APP by Western immunoblot did not reveal a significant difference between these mice (mean APP to actin ratio±1 SEM; Tg APP_{sw} mice, 1.16±0.06; Tg APP_{sw}/CD40L def. mice, 1.15±0.04), suggesting that the observed reduction of Aβ-β-amyloid in Tg APP_{sw}/CD40L def. mice was bit dye ti reduced APP production.

To evaluate whether CD40L deficiency might oppose gliosis in Tg APP_{sw} mice, we performed immunohistochemistry for detection of CD11b (a marker of activated microghliua) and glial fibrillary acidic protein (GFAP, increased in activated astrocytes). Microglial activation was reduced in Tg APP_{sw}/CD40L def. mice compared to Tg APP_{sw} mice in each of the three brain regions examined [cingulate cortex (CC), hippocampus (H), and entorhinal cortex (EC)] by 16 months of age. Quantitative image analysis revealed significant differences for each brain region, showing between 44% (CC) and 50% (EC) reduction in activated microglia. Examination of GFAP-positive astrocytes showed a similar pattern of results, with diminished astrocytic activation ranging from 30% (EC) to 46% (H). additionally, measurement of brain TVT-α protein [secreted by activated microglia and astrocytes] levels by Western immunoblot revealed a statistically significant (p<0.001) 64% reduction in Tg APP_{sw}/CD40L def. mice compared to Tg APP_{sw} mice (mean TNF-α to actin ratio±1 SEM: Tg APP_{sw} mice, 0.25±0.02; control littermates, 0.13±0.01; Tg APP_{sw}/CD40L def. mice, 0.09±01; CD40L def. mice, 0.09±0.02), providing further evidence of reduced gliosis in Tg APP_{sw}/CD40L def. mouse brains.

Anti-CD40L antibody was administered to a transgenic mouse model of AD. To expedite evaluation in these experiments, we administered anti-CD40L antibody to mice doubly transgenic for the “Swedish” APP and M146L PS1 mutations (PSAPP). These mice have previously been shown to produce copious β-amyloid deposits by 8 months of age (Holcomb et. “Accelerated Alzheimer-type phenotype in transgenic mice carrying both mutant amyloid precursor protein and presenilin 1 transgenes,” Nat. Med. (1998) 4:97-100). Anti-CD40L antibody was administered based on a treatment schedule previously described, which depletes CD40L in mice (Schonbeck et al., inhibition of CD40 signaling limits evolution of established atherosclerosis in mice,” Proc. Natl. Acad. Sci. USA (2000) 97:7458-63). At 8 months of age β-amyloid plaques appeared more diffuse in PSAPP mice that received anti-CD40L antibody treatment. Results revealed between 61% (H) and 74% (EC) reduction in β-amyloid plaques in PSAPP mice treated with anti-CD40L antibody versus isotype-matched control antibody. The largest reductions were observed in the hippocampus and entorhinal cortex, regions classically regarded to be most sensitive to AD pathology in humans (Schmidt et al. “Relative abundance of tau and neurofiliment epitopes in hippocampal neurofibrillary tangles,” Am. J. Pathol. (1990) 136:1069-75; Ball et al., “A new definition of Alzheimer's disease: a hippocampal dementia,” Lancet (1985) 1:14-16. Consistently, thioflavin S staining for β-amyloid revealed reductions of similar magnitude in these same regions. Thus, either genetic disruption of CD40L from conception or depletion of CD40L in adult transgenic mice results in mitigation of cerebral amyloidosis.

We examined the ratio of β-C-terminal fragment (β-CTF) to α-C-terminal fragment (α-CTF) of APP in Tg APP_{sw} mice, Tg APP_{sw}/CD40L def. mice, PSAPP animals treated with anti-CD40L antibody, and PSAPP mice treated with non-specific, isotype-matched control antibody. As previously reported, α-CTF and β-CTF were represented at similar levels in Tg APP_{sw} mice in contrast to the largely α-CTF processing of normal APP in murine cells (Leu et al., “mice deficient in BACE1, the Alzheimer's beta-secretase, have normal phenotype and abolished beta-amyloid generation,” Nat. Neurosci. (2001) 4:231-2). Strikingly, Tg APP_{sw}/CD40L def. animals had a marked decrease of β-CTF relative to α-CTF. In contrast to Tg APP_{sw} mice, in PSAPP animals, α-CTF was under-represented relative to β-CTF in animals that received non-relevant control IgG antibody (IgG-treated PSAPP mice did not differ from non-treated PSAPP animals, data not shown). This is consistent with the generation of excess Aβ-β-amyloid in these animals. By contrast, PSAPP mice that received anti-CD40L antibody manifested a shift in APP CTFs such that the ratio of β-CTF to α-CTF was markedly decreased compared to controls. To establish whether anti-CD40L antibody could penetrate the blood brain barrier and could potentially directly effect changes in CNS APP processing (as opposed to the generation of a peripheral signal or some o ther mechanism) we probed brain homogenates for hamster IgG antibody and found it to be present at 0.245% of circulating levels after 24 hours (no significant difference was found between anti-CD40L and control antibody, data not shown).

We have recently identified CD40 on neurons and neuron-like cells (including the N2a neuroblastoma cell line), and have shown that neuronal CD40 is functional, being intimately involved in neuronal development, survival, and maturation (Tan et al., “CD40 is expressed and functional on neuronal cells,” EMBO J. (2002) 21:643-52). Given our in vivo findings, we wished to determine whether CD40L could directly act on neurons to modulate APP processing. An N2a cell line was established that stably overexpresses (by ˜3 fold) the human wild-type APP-751 transgene (Xia et al., “Enhanced production and oligomerizatio of the 42-residue amyloid beta-protein by Chinese hamster ovary cells stably expressing mutant presenilins,” J. Biol. Chem. (1997) 272:7977-82). CD40L treatment of these cells results in a time-dependent decrease in α-CTF by Western blot. To confirm whether this reduction in α-CTF might be associated with amyloidogenic processing of APP, we measured secreted Aβ in conditioned media. Results show a time-dependent increase in both Aβ₁₋₄₀ and Aβ₁₋₄₂ levels, which is inversely related to α-CTF levels. Thus CD40L is able to directly promote amyloidogenic APP processing in neurons or neuron-like cells. Reducing the availability of CD40L in vivo has the opposite effect of adding CD40L in vitro on APP processing, both suggesting that CD40L regulates secretase cleavage of APP. As the vast majority of cases of AD are associated with accumulation of Aβ from a normal APP sequence, the observation that the processing of normal APP can be pushed towards amyloidogenicity by CD40L is of interest. In AD<it has been observed that an excess of CD40L-bearing astrocytes occurs (Calingasan et al., “Identification of CD40 ligand in Alzheimer's disease and in animal models of Alzheimer's disease and brain injury,” Neurobiol. Aging (2002) 23:31-9), and either membrane-bound or secreted forms of CD40L (Schonbeck et al., “The CD40/CD154 receptor/ligand dyad,” Cell Mol. Life Sci. (2001) 58:4-43) could influence cerebral APP processing towards Aβ formation.

Mice. CD40L deficient mice are the C57BL/6 background constructed as previously described (Xu et al., “Mice deficient for the CD40 ligand,” Immunity (1994) 1:423-31). Tg APP_{sw} mice are the 2576 line crossed with C57B6/SJL as previously described (Hsiao et al., “Correlative memory deficits, Abeta elevation, and amyloid plaques in transgenic mice,” Science (1996) 274:99-102). Also, CD40L deficient mice were crossded with Tg APP_{sw} transgenic mice and characterized offspring by polymerase chain reaction-based genotyping for the mutant APP construct (to examine Tg APP_{sw} status) and neomycin selection vector (to type for CD40L deficiency), followed by Western blot for brain APP and splenic CD40L protein, respectively. The animals that we studied at 12 and 16 months of age were Tg APP_{sw}/CD40L deficient (Tg APP_{sw}/CD40L def.; 12 months: 3 female, 16 months: 3 female/1 male), non-Tg APP_{sw}/CD40L deficient (CD40L def.; 12 months: 3 female, 16 months: 3 female/1 male), Tg APP_{sw}/CD40L wild-type(Tg APP_{sw}; 12 months: 3 female, 16 months: 2 female/1 male), and non-Tg APP_{sw}/CD40L wild-type control littermate mice (Control; 12 months: 3 female, 16 months: 2 female/1 male).

PSAPP were bred by crossing Tg APP_{sw} with PS1 M1467 mice as previously described (Holcomb et al., “Accelerated Alzheimer-type phenotype in transgenic mice carrying both mutant amyloid precursor protein and presenilin 1 transgenes,” Nat. Med. (1998) 4:97-100). A total of 10 PSAPP mice were used in this study, and 5 mice (3 female/2 male) received anti-CD40L IgG antibody (MR1), while the remaining 5 (2 female/3 male) received isotype-matched control IgG antibody. Beginning at 8 weeks of age, PSAPP mice were i.p. injected with 200 μg of the appropriate antibody once every ten days, based on previously described methods (Schonbeck et al., “Inhibition of CD40 signaling limits evolution of established atherosclerosis in mice,” Proc. Natl. Acad. Sci. USA (2000) 97:7458-63). These mice were then sacrificed at 8 months of age for analysis of Aβ deposits.

Mice were anesthetized with isofluorane and transcardinally perfused with ice-cold physiological saline containing heparin. Brains were rapidly dissected and quartered using a mouse brain slicer (Muromachi Kikai Co., Tokyo). The first and second anterior quarters were homogenized for Western blot analyses, and the third and fourth posterior quarters were used for microtome or cryostat sectioning. For microgliosis analysis, brains were quick-frozen at −80° C., and for β-amyloid immunohistochemistry, congo red staining, and astrocytosis, brains were immersed in 4% paraformaldehyde at 4° C. overnight, and routinely processed in paraffin. Five coronal sections from each brain (5 μm thickness) were cut with a 150 μm interval for these analyses. Immunohistochemical staining was performed in accordance with the manufacturer's instruction using the VECTASTAIN® Elite ABC kit (Vector Laboratories), except that, for CD11b staining, a biotinylated secondary mouse IgG absorbed anti-rat antibody was used in place of biotinylated anti-rabbit antibody that was supplied with the kit. Congo red staining was performed according to standard practice using 10% (w/v) filtered congo red dye cleared with alkaline alcohol. The following antibodies were variously employed for immunohistochemical staining: rabbit anti-cow GFAP antibody (1:500; DAKO), mouse anti-human amyloid-β antibody (4G8; 1:100; Signet), rabbit anti-human amyloid-β antibody (1:100; Sigma), and rat anti-mouse CD11b antibody (1:200; Caltag Laboratories).

Image analysis. Images were acquired from an Olympus BX60 microscope with an attached CCD video camera system (Olympus), and video signal was routed into a Windows 98SE™ PC via an AG5 averaging frame grabber (Scion Corporation) for quantitative analysis using Image-Pro software (Media Cybernetics). Images of five (5) μm sections (150 μm apart) through each anatomic region of interest (hippocampus or cortical areas) were captured and a threshold optical density was obtained that discriminated staining from background. Manual editing of each field was used to eliminate artifacts. For β-amyloid, congo red, and thioflavin S burden, and astrocytosis and microgliosis analyses, data are reported as the percentage of immunolabeled area captured (positive pixels) divided by the full area captured (total pixels). For β-amyloid plaque morphometric analysis, diameters of β-amyloid plaques were calculated via quantitative image analysis and numbers of plaques falling into each diameter category were totaled. Each immunohistochemical analysis was performed by a single examiner (T.M. or T.T.). Image analysis was performed prior to the revelation of sample identities.

ELISA analysis. Mouse brains (Control, Tg APP_{sw}, CD401 def., and Tg APP_{sw}/CD40L def.) were isolated under sterile conditions on ice and placed in ice-cold lysis buffer (containing 20 mM Tris, pH 7.5, 150 mM NaCl, 1 mM EDTA, 1 mM EGTA, 1% v/v Triton X-100, 2.5 mM sodium pyrophosphate, 1 mM β-glycerolphosphate, 1 mM Na₃VO₄, 1 μg/mL leupeptin, and 1 mM PMSF). Brains were then sonicated on ice for approximately 3 minutes, let stand for 15 minutes at 4° C., and centrifuged at 15,000 rpm for 15 minutes. Total Aβ species were detected by acid extraction of brain homogenates in 5 M guanidine buffer (Johnson-Wood et al., “Amyloid precursor protein processing and A beta42 deposition in a transgenic mouse model of Alzheimer disease,” Proc. Natl. Acad. Sci. USA (1997) 94:1550-55), followed by a 1:10 dilution in lysis buffer. Aβ₁₋₄₀, Aβ₁₋₄₂, and total Aβ (estimated by summing Aβ₁₋₄₀ and Aβ₁₋₄₂ values) were quantified in these samples using the Aβ₁₋₄₀ and Aβ₁₋₄₂ enzyme-linked immunosorbent assay (ELISA) kits (QCB) in accordance with the manufacturer's instruction, except that standards were diluted such that the final concentration included 0.5 M guanidine buffer. Total protein was quantified in brain homogenates using the Bio-Rad protein assay (Bio-Rad); thus, ELISA values are reported as ng of Aβ₁₋ₓ/wet g of brain. For in vitro analysis of Aβ levels, conditioned media from human APP-overexpressing N2a cells was collected and analyzed at a 1:1 dilution using the method described above, and values were reported as percentage of Aβ₁₋ₓ secreted relative to control.

Western blot. Mouse brains or cells were lysed in ice-cold lysis buffer as described abovce, and an aliquot corresponding to 50 μg of total protein was electrophoretically separated using 16.5% Tris-tricine gels (Bio-Rad, Hercules, Calif.). Electrophoresed proteins were then transferred to PVDF membranes (Bio-Rad), washed in dH₂0, and blocked for 1 h at ambient temperature in Tris-buffered saline (TBS) containing 5% (w/v) of non-fat dry milk. After blocking, membranes were hybridized for 1 h at ambient temperature with various antibodies against the C-terminus of APP or the N-terminus of Aβ. Membranes were then washed 3 times for 5 minutes each in dH₂0 and incubated for 1 hour at ambient temperature with the appropriate HRP-conjugated secondary antibody (1:1000, Santa Cruz Biotechnology, Santa Cruz, Calif.). All antibodies were diluted in TBS containing 5% (w/v) of non-fat milk. Blots were developed using the luminol reagent (Santa Cruz). Densitometric analysis was perfromed using the Fluor-S Multilmager™ with Quantity One™ software (Bio-Rad). Antibodies used for Western blot included antibody 369 (1:500, kindly provided by Dr. Sam Gandy), 6687 (1:1000, kindly provided by Dr. Harald Steiner), Chemicon anti-C-terminal APP antibody (1:500), BAM-10 (1:1000, Sigma), or actin (as an internal reference control, 1:1000, Roche, Germany).

Statistical analyses. All data for this example were found to be normally distributed; therefore, in instances of single mean comparison, Levene's test for equality of variances followed by t-Test for independent samples were used to assess significance. In instances of multiple mean comparisons, analysis of variance (ANOVA) was employed, followed by post-hoc comparison using Bonferroni's method. For all analyses, alpha levels were set at 0.05 and were performed using SPSS for Windows, release 10.0.5.

### EXAMPLE 3

### Detection of Phospho-Tau in Mouse Brain Sections

Immunohistochemistry. Transgenic mice [16 months old, including Tg APP_{sw} mice: n=4, 2 male/2 female, and Tg APP_{sw}/CD40L def. mice: n=5, 3 female, 2 male] were anesthetized with isofluorane and transcardinally perfused with ice-cold physiological saline containing heparin. Brains were rapidly dissected and immersed in 4% paraformaldehyde at 4° C. overnight. Brain tissue was routinely embedded in paraffin and processed according to standard practice. Five coronal sections (5 μm thickness) were cut with a 150 μm interval using a Reichert-Jung 2030 microtome (Leica Co., Nussloch, Germany). Immunohistochemical staining was performed in accordance with the manufacturer's instruction using the VECTASTAIN® Elite avadin biotin complex (ABC) kit (Vector Laboratories, Burlingame, Calif.). The primary antibodies that were employed were anti-phospho-tau S199 (1:50) and anti-phospho-tau S202 (1:200) (both antibodies were obtained from BioSource International, Camarillo, Calif.). Slides were permanently mounted and viewed under bright-field using an Olympus BX-60 microscope.

Image analysis. Bright-field images were acquired from an Olympus BX-60 microscope with an attached MagnaFire™ camera, and video signal was routed into a Windows 98SE™ PC for quantitative analysis using Image-Pro software (Media Cybernetics, Silver Spring, Md.). Images of five (5) μm sections (150 μm apart) through each anatomic region of interest (hippocampus or cortical areas) were captured and a threshold optical density was obtained that discriminated staining from background. Manual editing of each field was used to eliminate artifacts. Positive immunolabeled area was determined by dividing the percentage of immunolabeled area captured (positive pixels) by the full area captured (total pixels). Image analysis was performed in a blind fashion prior to the revelation of sample identities.

Results. Phoisphorylation of tau was examined in situ at 16 months of age in these mice using antibodies that recognize epitopes which are phosphorylated in AD brain (Genis et al., 1999). Antibody pS199 revealed numerous positive neurons, particularly in close vicinity of β-amyloid deposits in the neocortex and hippocampus of Tg APP_{sw} mice. Yet, in similar regions of Tg APP_{sw}/CD40L def mouse brains, this neuronal signal was either completely absent or markedly reduced. Quantitative image analysis of multiple brain sections revealed an 83% reduction in neocortical pS199 immunostaining, and a 70% reduction in hippocampal pS199 immunoreactivity. The t-Test for independent samples revealed significant differences between Tg APP_{sw} and Tg APP_{sw}/CD40L def. mice for the neocortex (p<0.01) and the hippocampus (p<0.05). Immunostaining was also performed using antibody pS202. The pattern of immunoreactivity for this antibody was quite different from that of pS199, as pS202 revealed a punctate staining pattern within the area delineated by the β-amyloid deposit, while pS202-positive neurons surrounding the β-amyloid deposit were few in number in both the neocortex and the hippocampus of Tg APP_{sw} mice. When comparing Tg APP_{sw} mice to Tg APP_{sw}/CD40L def. animals, pS202 immunoreactivity was markedly reduced in the latter group. Quantitative image analysis of multiple brain sections revealed a 95% reduction in neocortical pS202 immunostaining, and an 86% reduction in hippocampal pS202 immunoreactivity. The t-Test for independent samples revealed significant differences between Tg APP_{sw} and Tg APP_{sw}/CD40L def. mice for the neocortex (p<0.01) and the hippocampus (p<0.05). Phospho-tau as detected by pS199 or pS202 antibody was essentially absent in Tg APP_{sw} control littermates or CD40L def mice (data not shown).

### EXAMPLE 4

### Impact of Reducing CD40L Availability on Aβ-β-amyloid Pathology

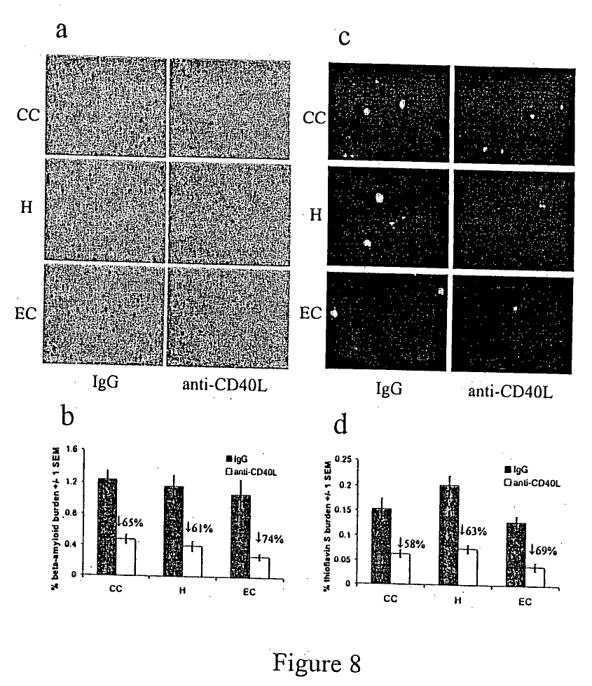

To evaluate the effects that reduction of functional CD40L in adult mice has for β-amyloid pathology, we administered anti-CD40L antibody to a transgenic mouse model of AD. To expedite evaluation in these experiments, we treated mice doubly transgenic for the “Swedish” APP and M146L PS1 mutations (PSAPP). These mice have previously been shown to produce copious β-amyloid deposits by 8 months of age (Holcomb, L. et al., “Accelerated Alzheimer-type phenotype in transgenic mice carrying both mutant amyloid precursor protein and presenilin 1 transgenes,” Nat. Med. 4, 97-100 (1998)). Anti CD40L antibody was administered based on a treatment schedule previously described, which depletes CD40L in mice (Schonbeck, U. et al., “Inhibition of CD40 signaling limits evolution of established atherosclerosis in mice.” Proc. Natl. Acad. Sci. USA 97, 7458-7463 (2000)). At 8 months of age β-amyloid plaques appeared more diffuse in PSAPP mice that received anti-CD40L antibody treatment (FIG. 8a). Results revealed between 61% (H) and 74% (EC) reduction in β-amyloid plaques in PSAPP mice treated with anti-CD40L antibody versus IgG control antibody (FIG. 8b). Consistently, thioflavin S staining for β-amyloid revealed reductions of similar magnitude (FIGS. 8c and 8d), with the largest alleviations in the hippocampus and entorhinal cortex, regions classically regarded to be most sensitive to AD pathology in humans (Schmidt, M. L., et al., “Relative abundance of tau and neurofilament epitopes in hippocampal neurofibrillary tangles,” Am. J. Pathol. 136, 1069-1075 (1990); Ball, M. J., et al., “A new definition of alzheimer's disease: a hippocampal dementia,” Lancet 1, 14-16 (1985)).

Aβ ELISA analysis of these animals' brains produced results consistent with the above findings [mean Aβ (ng/wet g of brain)±1 SEM of control IgG vs. anti-CD40L treated PSAPP mice; 34% reduction in Aβ₁₋₄₀: 1845.1±47.6 vs. 1.222.71±76.0; 47% reduction in Aβ₁₋₄₂: 2235.8±142.6 vs. 1179.0±82.5; 41% reduction in total Aβ: 4081.0±142.1 vs. 2401.7±154.0, P<0.001 for each comparison]. As with Tg APP_{sw}/CD40L def. mice compared to Tg APP_{sw} animals, reductions in Aβ/β-amyloid pathology in anti-CD40L antibody versus control IgG-treated PSAPP mice were generally associated with reduced activation of microglia observed by CD11b immunostaining and image analysis (particularly in the H, 59% reduction, P<0.01; in the EC, 47% mitigation, P<0.05; in the CC, no significant differences). Additionally, reactive astrocytes (by GFAP immunostaining and image analysis) were reduced in these same animals (in the H, 51% decrease, P<0.01; in EC, 83% reduction, P<0.001; in the CC, 71% mitigation, P<0.001). Thus, either genetic disruption of CD40L from conception, or depletion of CD40L in adult transgenic mice resulted in mitigation of gliosis and cerebral amyloidosis.

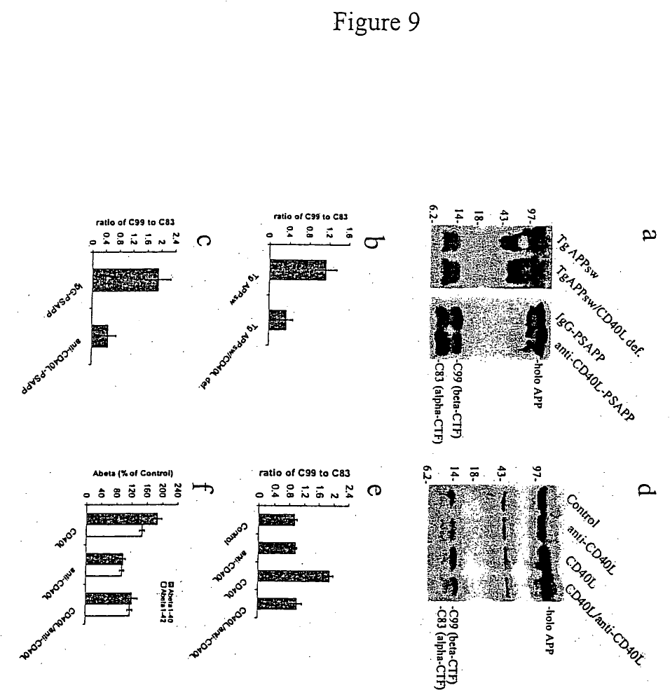

To determine whether reduction of available CD40L had an effect on APP metabolism, we examined the ratio of APP β-C-terminal fragment (β-CTF) to α-C-terminal fragment (α-CTF) in Tg APP_{sw} mice, in Tg APP_{sw}/CD40L def. mice, PSAPP animals treated with anti-CD40L antibody, or PSAPP mice treated with IgG control antibody. As previously reported, α-CTF and β-CTF were rerpresented at similar levels in Tg APP_{sw} mice in contrast to the largely α-CTF processing of normal APP in murine cells (Luo, Y. et al., “Mice deficient in BACE1, the Alzheimer's beta-secretase, have normal phenotype and abolished beta-amyloid generation,” Nat. Neurosci. 4, 231-232 (2001)). Strikingly, Tg APP_{sw}/CD40L def. animals had a marked decrease in β-CTF relative to α-CTF compared to Tg APP_{sw} mice (FIGS. 9a and 9b). This shift from amyloidogenic to non-amyloidogenic APP processing in Tg APP_{sw}/CD40L def. mice versus Tg APP_{sw} mice was accompanied by significant decreases in β- and γ-secretase cleavage activity as determined by APP secretase cleavage activity assay [mean (%) reduction±1 SEM (%), 46.54±5.87 and 31.21±7.44 reductions in β- and γ-secretase activities, respectively]. PSAPP mice that received anti-CD40L antibody manifested a shift in APP CTFs such that the ratio of β-CTF to α-CTF was markedly decreased compared to control IgG antibody-treated mice (FIGS. 9a and 9c).

To establish whether anti-CD40L antibody could penetrate the blood brain barrier and could potentially directly effect changes in CNS APP processing (as opposed to the generation of a pheripheral signal or some other mechanism) we probed brain homogenates for hamster IgG antibody and found it to be present at 0.245% of circulating levels after 24 hours (no significant difference was found between anti-CD40L and IgG control antibody, data not shown). These data suggest that the reduction of available CD40L mitigates Aβ-β-amyloid pathology by the shifting of APP processing from the amyloidogenic to the non-amyloidogenic pathway.

We also employed an N2a cell line that stably over-expresses the human wild-type APP-695 transgene (Thinakaran, G., et al., “Metabolism of the Swedish’ amyloid precursor protein variant in neuro2a (N2a) cells, Evidence that cleavage at the ‘beta-secretase’ site occurs in the golgi apparatus,” J. Biol. Chem. 271, 9390-9397 (1996)). CD40L treatment of these cells under serum-free conditions for 24 hours resulted in an increased ratio of APP β-CTF to α-CTF by Western blot (FIGS. 9d and 9e). This effect could be alleviated by co-treatment with anti-CD40L antibody, as we detected anti-CD40L antibody in brains of treated PSAPP mice. To determine whether an incrreased ratio of β-CTF to α-CTF after CD40L treatment might be associated with secretion of Aβ, we measured the latter in conditioned transfected N2a cell media by ELISA. Results showed approximate 85% and 50% increases in Aβ₁₋₄₀ and Aβ₁₋₄₂ levels, respectively, after 24 hour treatment with CD40L, an effect that could be blocked by co-treatment with anti-CD40L antibody (FIG. 9f). To confirm the specificity of this effect, we treated these cells with other TNF ligand superfamily members TNF-α or Fas ligand the additional control ligands transforming growth factor-β1 or neurotrophin. We did not observe alterations in APP CTFs or in secretion of Aβ species following treatment with these ligand controls (data not shown).

Having established in vitro that CD40L challenge was able to promote Aβ production, and that depleting CD40L shifted APP metabolism from amyloidogenic to non-amyloidogenic in vivo, we examined if reducing available CD40L could additionally affect clearance of Aβ. We were particularly interested in this possibility as vascular endothelial and smooth muscle cells express CD40 (Schonbeck, U. et al., “Ligation of CD40 activates interleukin-1beta-converting enzyme (caspase-1) activity in vascular smooth muscle and endothelial cells and promotes elaboration of active interleukin 1 beta,” J. Biol. Chem. 272, 19569-19574 (1997); Mach, F. et al. “Functional CD40 ligand is expressed on human vascular endothelial cells, smooth muscle cells, and macrophages: implications for CD40-CD40 ligand singaling in atherosclerosis,” Proc. Natl. Acad. Sci. USA 94, 1931-1936 (1997)), and it has been shown that CD40L signaling is able to modulate blood-brain-barrier premeability in mice (Piguet, P. F. et al., “Role of CD40-CD49L in mouse severe malaria,” Am. J. Pathol. 159, 733-742 (2001)). Movement of Aβ from brain to blood has recently been found after a treatment strategy involving passive immunization with anti-Aβ antibodies (DeMattos, R. B. et al., “Peripheral anti-A beta antibody alters CNS and plasma A beta clearance and decreases brain A beta burden in a mouse model of Alzheimer's disease,” Proc. Natl. Acad. Sci. USA 98, 8850-8855 (2001); DeMattos, R. B., et al., “Brain to plasma amyloid-beta efflux: a measure of brain amyloid burden in a mouse model of Alzheimer's disease,” Science 295, 2264-2267 (2002)).

To determine if anti-CD40L antibody could promote brain-to-blood clearance of Aβ, we obtained blood plasma from PSAPP mice treated with anti-CD40L antibody or from animals given IgG control antibody. Strikingly, data showed marked increases in Aβ₁₋₄₀ and Aβ₁₋₄₂ levels in blood plasma from 8 month old PSAPP mice treated with anti-CD40L antibody [mean Aβ (pg/mL)±1 SEM of control IgG vs. anti-CD40L treated PSAPP mice; 77% increase in Aβ₁₋₄₀: 304.9±36.3 vs. 1334.9±171.6; 77% increase in Aβ₁₋₄₂: 77.5±25.9 vs. 335.7±42.9; 80% increase in total Aβ: 382.4±62.2 vs. 1899.2±318.8, P<0.001 for each comparison]. Treatment of another cohort of PSAPP mice with a single injection of anti-CD40L or control IgG antibody (n=9 for each condition, 5 male/4 female) revealed that this effect was first observable at 24 hours post-injection (data not shown). Thus, anti-CD40L antibody treatment promoted increased circulating levels of Aβ concomitant with decreased CNS levels, suggesting brain-to-blood clearance of Aβ.

Our data show that genetic ablation of the CD40L gene or pharmacologic reduction of available CD40L both resulted in amelioration of Aβ/β-amyloid pathology. In addition to attenuating gliosis, reduction of available CD40L was able to shift APP metabolism from the amyloidogenic to the non-amylodogenic pathway in vivo. Reducing the availability of CD40L in vivo had the opposite effect on APP processing of adding CD40L to neuron-like cells in vitro, both indicating that CD40L signaling regulated secretase clevage of APP. This is supported by the observation that in the Tg APP_{sw} mice CD40L deficiency was associated with decreases in total brain β- and γ-secretase activities. As the vast majority of cases of AD are associated with accumulation of Aβ from a normal APP sequence, the observation that the processing of normal APP could be pushed towards amyloidogenicity by CD40L is of interest. Finally, administration of anti-CD40L antibody to PSAPP mice resulted in increased plasma levels of Aβ concomitant with reduced cerebral Aβ/β-amyloid pathology, suggesting that depletion of CD40L promoted brain clearance of Aβ. Thus, strategies aimed at reducing available CD40L are able to reduce Aβ/β-amyloid pathology via multiple mechanisms.

Immunohistochemistry and morphometry (FIGS. 8-9). Mice were anesthetized with isoflurane and transcardinally perfused with ice-cold physiological saline containing heparin. Brains were rapidly dissected and quartered using a mouse brain slicer (Muromachi Kikai Co., Tokyo, Japan). This first and second anterior quarters were homogenized for Western blot analyses, and the third and fourth posterior quarters were used for microtome or cryostat sectioning. For microgliosis analysis, brains were quick-frozen at −80° C., and for β-amyloid immunohistochemistry, congo red staining, and astrocytosis, brains were immersed in 4% paraformaldehyde at 4° C. overnight, and routinely processed in paraffin. Five coronal sections from each brain (5 μm thickness) were cut with a 150 μm interval for these analyses. Immunohistochemical staining was performed in accordance with the manufacturer's instruction using the VECTASTAIN® Elite ABC kit (Vector Laboratories, Burlingame, Calif., USA), except that, for CD11b staining, a biotinylated secondary mouse IgG absorbed anti-rat antibody was used in place of the biotinylated anti-rabbit antibody that was supplied with the kit. Congo red staining was performed according to standard practice using 10% (w/v) filtered congo red dye cleared with alkaline alcohol. The following antibodies were variously employed for immunohistochemical staining: rabbit anti-cow GFAP antibody (1:500; DAKO, Carpintria, Calif.), mouse anti-human amyloid-β antibody (4G8; 1:100; Signet, Dedham, Mass.), rabbit anti-human amyloid-β antibody (1:100; Signma, Saint Louis, Mo., USA), and rat anti-mouse CD11b antibody (1:200; Caltag Laboratories, Burlingame, Calif., USA).

Image analysis (FIGS. 8-9). Images were acquired from an Olympus BX60 microscope with an attached CCD video camera system (Olympus America Inc., Melville, N.Y., USA), and video signal was routed into a Windows 98SE™ PC via an AG5 averaging frame grabber (Scion Corporation, Frederick, Md., USA) for quantitative analysis using Image-Pro software (Media Cybernetics, Carlsbad, Calif., USA). Images of five (5) μm sections (150 μm apart) through each anatomic region of interest (hippocampus or cortical areas) were captured and a threshold optical density was obtained that discriminated staining from background. Manual editing of each field was used to eliminate artifacts. For β-amyloid, congo red, and thioflavin S burden, and astrocytosis and microgliosis analyses, data are reported as the percentage of immunolabeled area captured (positive pixels) divided by the full area captured (total pixels). For β-amyloid plaque morphometric analysis, diameters of β-amyloid plaques were calculated via quantitative image analysis and numbers of plaques falling into each diameter category were totaled. Image analysis was performed prior to the revelation of sample identities.

ELISA analysis (FIGS. 8-9). Mouse brains (Control, Tg APP_{sw}, CD40L def., and Tg APP_{sw}/CD40L def.) were isolated under sterile conditions on ice and placed in ice-cold lysis buffer (containing 20 mM Tris, pH 7.5, 150 mM NaCl, 1 mM EDTA, 1 mM EGTA, 1% v/v Triton X-100, 2.5 mM sodium pyrophosphate, 1 mM β-glycerolphosphate, 1 mM Na₃VO₄, 1 pg/mL leupeptin, and 1 mM PMSF). Brains were then sonicated on ice for approximately 3 minutes, let stand for 15 minutes at 4° C., and centrifuged at 15,000 rpm for 15 minutes. Total Aβ species were detected by acid extraction of brain homogenates in 5 M guanidine buffer (Johnson-Wood, K. et al., “Amyloid precursor protein processing and A beta42 deposition in a transgenic mouse model of Alzheimer disease,” Proc. Natl. Acad. Sci USA 94, 1550-1555 (1997)), followed by a 1:10 dilution in lysis buffer. Aβ₁₋₄₀ and Aβ₁₋₄₂ and total Aβ (estimated by summing Aβ₁₋₄₀ and Aβ₁₋₄₂ values) were quantified in these samples using the Aβ₁₋₄₀ and Aβ₁₋₄₂ enzyme-linked immunosorbent assay (ELISA) kits (BioSource, Camarillo, Calif., USA) in accordance with the manufacurer's instruction, except that standards were diluted such that the final concentration included 0.5 M guanidine buffer. Total protein was quantified in brain homogenates using the Bio-Rad protein assay (Bio-Rad, Richmond, Calif., USA); thus, ELISA values were reported as ng of Aβ₁₋ₓ/wet g of brain. For in vitro analysis of Aβ levels, conditioned media from human APP-over-expressing N2a cells were collected and analyzed at a 1:1 dilution using the method described above, and values were rreported as percentage of Aβ₁₋ₓ secreted relative to control. Blood plasma was used neat at a 1:4 dilution using the method described above for determination of plasma Aβ levels, and values were reported as pg/mL of Aβ₁₋ₓ.

Western blot (FIGS. 8-9). Mouse brains or cultured cells were lysed in ice-cold lysis buffer as described above, and an aliquot corresponding to 50 μg of total protein was electrophoretically separated using 16.5% Tris-tricine gels (Bio-Rad). Electrophoresed proteins were then transferred to PVDF membranes (Bio-Rad), washed in dH₂0, and blocked for 1 hour at ambient temperature in Tris-buffered saline (TBS) containing 5% (w/v) of non-fat dry milk. After blocking membranes were hybridized for 1 hour at ambient temperature with various antibodies against the C-terminus of APP or the N-terminus of Aβ. Membranes were then washed 3 times for 5 minutes each in dH₂0 and incubated for 1 hour at ambient temperature with the appropriate HRP-conjugated secondary antibody (1:1000, Santa Cruz Biotechnology, Santa Cruz, Calif., USA). All antibodies were diluted in TBS containing 5% (w/v) of non-fat dry milk. Blots were developed using the luminol reagent (Santa Cruz Biotechnology). Densitometric analysis was performed using the Fluor-S Multilmager™ with Quantity One™ software (Bio-Rad). Antibodies used for Western blot included antibody 369 (1:500), 6687 (1:1000), anti-C-terminal APP antibody (1:500; Chemicon, Temecula, Calif., USA), BAM-10 (1:1000, Sigma), or actin (as an internal reference control, 1:1000, Roche, Basel, Switzerland). Further β- and γ-secretase activities were quantified in Tg APP_{sw} and Tg APP_{sw}/CD40L def. mice using available kits based on secretase-specific peptides conjugated to fluorgenic reporter molecules (R&D Systems, Minneapolis, Minn., USA). All data were found to be normally distributed; therefore, in instances of single mean comparison, Levene's test for equality of variances followed by t Test for independent samples was used to assess significance. In instances of multiple mean comparisons, analysis of variance (ANOVA) was employed, followed by post-hoc comparison using Bonferroni's method. For all analyses, alpha levels were set at 0.05. All analyses were performed using SPSS for Windows™, Release 10.0.5 (SPSS Inc., Chicago, Ill., USA).

All patents, patent application, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

The invention claimed is:

1. A research model for screening compounds suspected of modulating the CD40L/CD40R signaling pathway by interfering with the CD40L/CD40R signaling pathway in an animal, human, or system, comprising the following steps:
contacting a first sample of cells with CD40 ligand and measuring the level or amount of a marker;
contacting a second sample of cells with a compound and CD40 ligand and measuring the level or amount of a marker; and
comparing the level or amount of the marker of the first sample of cells with the level or amount of the marker of the second sample of cells.

2. The method of claim 1, wherein the sample of cells are central nervous system cells, cell lines derived from central nervous system cells, peripheral cells, cell lines derived from peripheral cells, transgenic cells, transgenic cells derived from transgenic animals, human cells or cell lines, or immortalized or non-immortalized cell lines derived from humans, higher primates, primates or murine sources.

3. The method of claim 1, wherein the marker is the levels or amounts of one or more cytokines.

4. The method of claim 3, wherein the cytokine is selected from the group consisting of tumor necrosis factor, interleukin 1, interleukin 6, interleukin 12, interleukin 18, macrophage inflammatory protein, macrophage chemoattractant protein, granulocyte-macrophage colony stimulating factor, macrophage colony stimulating factor, and combinations thereof.

5. The method of claim 1, wherein the marker is selected from the group consisting of levels, amounts or activities of glutamate release, nitric oxide production, nitric oxide synthase, superoxide, superoxide dismutase, glutamate release, nitric oxide production, nitric oxide synthase, superoxide, superoxide dismutase, and combinations thereof.

6. The method of claim 1, wherein the marker is selected from the group consisting of a major histocompatability complex molecule, CD45, CD11b, F4/80 antigen, integrins, a cell surface molecule, and combinations thereof.

7. The method of claim 1, wherein the marker is decreased neuronal inflammation, the levels or amounts of Aβ, β-amyloid precursor protein (β-APP), a fragment of β-APP, a fragment of Aβ, or combinations thereof.

8. The method of claim 1, wherein the compound is a compound that binds to CD40R, a compound that binds to CD40L, a compound that decreases trimerization of CD40R, a compound that decreases trimerization of CD40L, a compound that modulates the CD40L/CD40R signaling pathway upstream or downstream of CD40L/CD40R interaction, a compound that reduces the phosphorylation of the tau protein or mutants thereof, a compound that interferes with TNF receptor-associated factors, a compound that interferes with presenilin-1 and/or presenilin-2, a compound that inhibits β-secretase activity, a compound that inhibits γ-secretase activity, a compound that enhances α-secretase activity, a compound that alters APP processing, a compound that reduces the ratio of APP β-CTF to APP α-CTF, a compound that reduces the amount of β-CTF, a compound that promotes brain-to-blood clearance of β-amyloid, a compound that increases circulating levels of β-amyloid, a compound that reduces the size and/or number of amyloid plaques, a compound that reduces β-amyloid burden, a compound that reduces soluble β-amyloid levels, a compound that reduces total β-amyloid levels, a compound that reduces congophilic β-amyloid deposits, a compound that reduces reactive gliosis, microgliosis, astrocytosis and combinations thereof, a soluble CD40R compound, a soluble CD40L compound, an immunogenic CD40L compound, a soluble CD40L variant (CD40LV) compound, an interfering RNA (dsRNA, RNAi or siRNA) compound to CD40R, an antisense RNA compound to CD40R, an interfering RNA (dsRNA, RNAi or siRNA) compound to CD40L, an antisense RNA compound to CD40L or combinations of interfering RNA (dsRNA, RNAi or siRNA) compounds and antisense compounds, or a compound selected from the group consisting of agonistic antibodies to CD40R, agonistic antibodies to CD40L, antagonistic antibodies to CD40R and antagonistic antibodies to CD40L.

9. The method of claim 8, wherein the interfering RNA (dsRNA, RNAi or siRNA) comprises polynucleotide sequences identical or homologous to CD40L, CD40R or β-amyloid.

10. The method of claim 9, wherein the interfering RNA (dsRNA, RNAi or siRNA) has greater than 70% homology to CD40L, CD40R or β-amyloid.

11. The method of claim 9, wherein the interfering RNA (dsRNA, RNAi or siRNA) has greater than 95% homology to CD40L, CD40R or β-amyloid.

12. The method of claim 8, wherein gene fragments of CD40L, CD40R or β-amyloid are targeted with the interfering RNA (dsRNA, RNAi or siRNA), and wherein the gene fragments are approximately 15-25 nucleotides in length.

13. The method of claim 8, wherein gene fragments of CD40L, CD40R or β-amyloid are targeted with the interfering RNA (dsRNA, RNAi or siRNA), and wherein the gene fragments are greater than 25 nucleotides in length.

14. The method of claim 8, wherein gene fragments of CD40L, CD40R or β-amyloid are targeted with the interfering RNA (dsRNA, RNAi or siRNA), and wherein the gene fragments are greater than 50 nucleotides in length.

15. The method of claim 8, wherein gene fragments of CD40L, CD40R or β-amyloid are targeted with the interfering RNA (dsRNA, RNAi or siRNA), and wherein the gene fragments are one nucleotide less than the full length gene of CD40L, CD40R or β-amyloid.

16. The method of claim 1, wherein the animal or human is afflicted with a disease or disorder selected from the group consisting of neuronal inflammation, brain injury, brain trauma, tauopathy, and an amyloidogenic disease.

17. The method of claim 16, wherein the amyloidogenic disease is selected from the group consisting of Alzheimer's disease, scrapie, transmissible spongiform encepalopathy, hereditary cerebral hemorrhage with amyloidosis Icelandic-type, hereditary cerebral hemorrhage with amyloidosis Dutch-type, familial Mediterranean fever, familial amyloid nephropathy with urticaria and deafness (Muckle-Wells syndrome), myeloma or macroglobulinemia-associated idiopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes Type II, and insulinoma.

18. The method of claim 16, wherein the tauopathy is selected from the group consisting of Alzheimer's disease, frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amyotrophy complex, Pick's disease, and Pick's disease-like dementia.

19. The method of claim 1, wherein the animal is a non-transgenic animal or a transgenic animal selected from the group consisting of worms, flies or mice.

20. The method of claim 19, wherein the transgenic animal expresses transgenic APP, overexpresses trangsgenic presenilin protein, overexpresses CD40R, overexpresses trangenic CD40L, and/or expresses tau protein or mutants thereof.

21. A research model for screening compounds suspected of modulating the CD40L/CD40R signaling pathway by interfering with the CD40L/CD40R signaling pathway in an animal, human, or system, comprising the following steps:
contacting CNS cells expressing CD40R with CD40L and a compound and measuring a marker;
contacting peripheral cells expressing CD40R with CD40L and the compound and measuring a marker;
contacting CNS cells with a stimulator of the CD40L/CD40R signaling pathway and the compound and measuring a marker;
contacting peripheral cells with a stimulator of the CD40L/CD40R signaling pathway and the compound and measuring a marker;
contacting CNS cells with an inhibitor of the CD40L/CD40R signaling pathway and the compound and measuring a marker;
contacting peripheral cells with an inhibitor of the CD40L/CD40R signaling pathway and the compound and measuring a marker;
comparing the markers to identify those compounds that modulate the CD40L/CD40R signaling pathway.

22. The method of claim 21, wherein the marker is the levels or amounts of one or more cytokines.

23. The method of claim 22, wherein the cytokine is selected from the group consisting of tumor necrosis factor, interleukin 1, interleukin 6, interleukin 12, interleukin 18, macrophage inflammatory protein, macrophage chemoattractant protein, granulocyte-macrophage colony stimulating factor, macrophage colony stimulating factor, and combinations thereof.

24. The method of claim 21, wherein the marker is selected from the group consisting of levels, amounts or activities of glutamate release, nitric oxide production, nitric oxide synthase, superoxide, superoxide dismutase, glutamate release, nitric oxide production, nitric oxide synthase, superoxide, superoxide dismutase, and combinations thereof.

25. The method of claim 21, wherein the marker is selected from the group consisting of a major histocompatability complex molecule, CD45, CD11b, F4/80 antigen, integrins, a cell surface molecule, and combinations thereof.

26. The method of claim 21, wherein the marker is decreased neuronal inflammation, the levels or amounts of Aβ, β-amyloid precursor protein (β-APP), a fragment of β-APP, a fragment of Aβ, or combinations thereof.

27. The method of claim 21, wherein the compound is a compound that binds to CD40R, a compound that binds to CD40L, a compound that decreases trimerization of CD40R, a compound that decreases trimerization of CD40L, a compound that modulates the CD40L/CD40R signaling pathway upstream or downstream of CD40L/CD40R interaction, a compound that reduces the phosphorylation of the tau protein or mutants thereof, a compound that interferes with TNF receptor-associated factors, a compound that interferes with presenilin-1 and/or presenilin-2, a compound that inhibits β-secretase activity, a compound that inhibits γ-secretase activity, a compound that enhances α-secretase activity, a compound that alters APP processing, a compound that reduces the ratio of APP β-CTF to APP α-CTF, a compound that reduces the amount of β-CTF, a compound that promotes brain-to-blood clearance of β-amyloid, a compound that increases circulating levels of β-amyloid, a compound that reduces the size and/or number of amyloid plaques, a compound that reduces β-amyloid burden, a compound that reduces soluble β-amyloid levels, a compound that reduces total β-amyloid levels, a compound that reduces congophilic β-amyloid deposits, a compound that reduces reactive gliosis, microgliosis, astrocytosis, and combinations thereof, a soluble CD40R compound, a soluble CD40L compound, an immunogenic CD40L compound, a soluble CD40LV compound, an interfering RNA (dsRNA, RNAi or siRNA) compound to CD40R, an antisense RNA compound to CD40R, an interfering RNA (dsRNA, RNAi or siRNA) compound to CD40L, an antisense RNA compound to CD40L or combinations of interfering RNA (dsRNA, RNAi or siRNA) compounds and antisense compounds, or a compound selected from the group consisting of agonistic antibodies to CD40R, agonistic antibodies to CD40L, antagonistic antibodies to CD40R and antagonistic antibodies to CD40L.

28. The method of claim 27, wherein the interfering RNA (dsRNA, RNAi or siRNA) comprises polynucleotide sequences identical or homologous to CD40L, CD40R or β-amyloid.

29. The method of claim 28, wherein the interfering RNA (dsRNA, RNAi or siRNA) has greater than 70% homology to CD40L, CD40R or β-amyloid.

30. The method of claim 28, wherein the interfering RNA (dsRNA, RNAi or siRNA) has greater than 95% homology to CD40L, CD40R or β-amyloid.

31. The method of claim 27, wherein gene fragments of CD40L, CD40R or β-amyloid are targeted with the interfering RNA (dsRNA, RNAi or siRNA), and wherein the gene fragments are approximately 15-25 nucleotides in length.

32. The method of claim 27, wherein gene fragments of CD40L, CD40R or β-amyloid are targeted with the interfering RNA (dsRNA, RNAi or siRNA), and wherein the gene fragments are greater than 25 nucleotides in length.

33. The method of claim 27, wherein gene fragments of CD40L, CD40R or β-amyloid are targeted with the interfering RNA (dsRNA, RNAi or siRNA), and wherein the gene fragments are greater than 50 nucleotides in length.

34. The method of claim 27, wherein gene fragments of CD40L, CD40R or β-amyloid are targeted with the interfering RNA (dsRNA, RNAi or siRNA), and wherein the gene fragments are one nucleotide less than the full length gene of CD40L, CD40R or β-amyloid.

35. The method of claim 21, wherein the animal or human is afflicted with a disease or disorder selected from the group consisting of neuronal inflammation, brain injury, brain trauma, tauopathy, and an amyloidogenic disease.

36. The method of claim 35, wherein the amyloidogenic disease is selected from the group consisting of Alzheimer's disease, scrapie, transmissible spongiform encepalopathy, hereditary cerebral hemorrhage with amyloidosis Icelandic-type, hereditary cerebral hemorrhage with amyloidosis Dutch-type, familial Mediterranean fever, familial amyloid nephropathy with urticaria and deafness (Muckle-Wells syndrome), myeloma or macroglobulinemia-associated idiopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes Type II, and insulinoma.

37. The method of claim 35, wherein the tauopathy is selected from the group consisting of Alzheimer's disease, frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amyotrophy complex, Pick's disease, and Pick's disease-like dementia.

38. The method of claim 21, wherein the animal is a non-transgenic animal or a transgenic animal selected from the group consisting of worms, flies or mice.

39. The method of claim 38, wherein the transgenic animal expresses transgenic APP, overexpresses trangsgenic presenilin protein, overexpresses CD40R, overexpresses trangenic CD40L, and/or expresses tau protein or mutants thereof.

40. A method of identifying compounds and/or small molecules that reduce, ameliorate, or modulate signs and/or symptoms associated with neuronal inflammation, brain injury, brain trauma, tauopathies, or amyloidogenic diseases, comprising administering a compound that modulates the CD40L/CD40R signaling pathway to an animal, human or system and measuring or observing the reduction, amelioration, or modulation of the symptoms.

41. The method of claim 40, wherein the compounds and/or small molecules are compounds and/or small molecules that bind to CD40R, compounds and/or small molecules that bind to CD40L, compounds and/or small molecules that decrease trimerization of CD40R, compounds and/or small molecules that decrease trimerization of CD40L, compounds and/or small molecules that modulate the CD40L/CD40R signaling pathway upstream or downstream of CD40L/CD40R interaction, compounds and/or small molecules that reduce the phosphorylation of the tau protein or mutants thereof, compounds and/or small molecules that interfere with TNF receptor-associated factors, compounds and/or small molecules that interfere with presenilin-1 and/or presenilin-2, compounds and/or small molecules that inhibit β-secretase activity, compounds and/or small molecules that inhibit γ-secretase activity, compounds and/or small molecules that enhance α-secretase activity, compounds and/or small molecules that alter APP processing, compounds and/or small molecules that reduce the ratio of APP β-CTF to APP α-CTF, compounds and/or small molecules that reduce the amount of β-CTF, compounds and/or small molecules that promote brain-to-blood clearance of β-amyloid, compounds and/or small molecules that increase circulating levels of β-amyloid, compounds and/or small molecules that reduce the size and/or number of amyloid plaques, compounds and/or small molecules that reduce β-amyloid burden, compounds and/or small molecules that reduce soluble β-amyloid levels, compounds and/or small molecules that reduce total β-amyloid levels, compounds and/or small molecules that reduce congophilic β-amyloid deposits, compounds and/or small molecules that reduce reactive gliosis, microgliosis, astrocytosis, and combinations thereof, a soluble CD40R compound, a soluble CD40L compound, an immunogenic CD40L compound, a soluble CD40LV compound, an interfering RNA (dsRNA, RNAi or siRNA) compound to CD40R, an antisense RNA compound to CD40R, an interfering RNA (dsRNA, RNAi or siRNA) compound to CD40L, an antisense RNA compound to CD40L or combinations of interfering RNA (dsRNA, RNAi or siRNA) compounds and antisense compounds, or a compound selected from the group consisting of agonistic antibodies to CD40R, agonistic antibodies to CD40L, antagonistic antibodies to CD40R and antagonistic antibodies to CD40L.

42. The method of claim 41, wherein the interfering RNA (dsRNA, RNAi or siRNA) comprises polynucleotide sequences identical or homologous to CD40L, CD40R or β-amyloid.

43. The method of claim 42, wherein the interfering RNA (dsRNA, RNAi or siRNA) has greater than 70% homology to CD40L, CD40R or β-amyloid.

44. The method of claim 42, wherein the interfering RNA (dsRNA, RNAi or siRNA) has greater than 95% homology to CD40L, CD40R or β-amyloid.

45. The method of claim 41, wherein gene fragments of CD40L, CD40R or β-amyloid are targeted with the interfering RNA (dsRNA, RNAi or siRNA), and wherein the gene fragments are approximately 15-25 nucleotides in length.

46. The method of claim 41, wherein gene fragments of CD40L, CD40R or β-amyloid are targeted with the interfering RNA (dsRNA, RNAi or siRNA), and wherein the gene fragments are greater than 25 nucleotides in length.

47. The method of claim 41, wherein gene fragments of CD40L, CD40R or β-amyloid are targeted with the interfering RNA (dsRNA, RNAi or siRNA), and wherein the gene fragments are greater than 50 nucleotides in length.

48. The method of claim 41, wherein gene fragments of CD40L, CD40R or β-amyloid are targeted with the interfering RNA (dsRNA, RNAi or siRNA), and wherein the gene fragments are one nucleotide less than the full length gene of CD40L, CD40R or β-amyloid.

49. The method of claim 48, wherein the signs and/or symptoms are a decrease in neuronal inflammation, a decrease in the trimerization of CD40R, a decrease in the trimerization of CD40L, modulation of the CD40L/CD40R signaling pathway upstream or downstream of CD40L/CD40R interaction, a reduction in the phosphorylation of the tau protein or mutants thereof, interference with TNF receptor-associated factors, interference with presenilin-1 and/or presenilin-2, that inhibition of β-secretase activity, inhibition of γ-secretase activity, enhancement of α-secretase activity, alteration of APP processing, reduction in the ratio of APP β-CTF to APP α-CTF, reduction in the amount of β-CTF, promotion of brain-to-blood clearance of β-amyloid, an increase in circulating levels of β-amyloid, or signs and/or symptoms selected from the group consisting of a reduction in the size and/or number of amyloid plaques, a reduction in β-amyloid burden, a reduction in soluble β-amyloid levels, a reduction in total β-amyloid levels, a reduction in congophilic β-amyloid deposits, a reduction in reactive gliosis, microgliosis, astrocytosis, and combinations thereof.

50. The method of claim 48, wherein the amyloidogenic disease is selected from the group consisting of Alzheimer's disease, scrapie, transmissible spongiform encepalopathy, hereditary cerebral hemorrhage with amyloidosis Icelandic-type, hereditary cerebral hemorrhage with amyloidosis Dutch-type, familial Mediterranean fever, familial amyloid nephropathy with urticaria and deafness (Muckle-Wells syndrome), myeloma or macroglobulinemia-associated idiopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes Type II, and insulinoma.

51. The method of claim 48, wherein the tauopathy is selected from the group consisting of Alzheimer's disease, frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amyotrophy complex, Pick's disease, and Pick's disease-like dementia.

52. The method of claim 48, wherein the animal is a non-transgenic animal or a transgenic animal selected from the group consisting of worms, flies or mice.

53. The method of claim 52, wherein the transgenic animal expresses transgenic APP, overexpresses transgenic presenilin protein, overexpresses CD40R, overexpresses transgenic CD40L, and/or expresses tau protein or mutants thereof.

54. A method of treating neuronal inflammation, brain injury, brain trauma, tauopathies, amyloidogenic diseases, or internal organ diseases related to amyloid plaque formation, in an individual, comprising administering to an individual therapeutically effective amounts of a composition comprising a carrier and an agent that interferes with the CD40L/CD40R signaling pathway or the phosphorylation of tau protein.

55. The method of claim 54, wherein the agent is a compound that binds to CD40R, a compound that binds to CD40L, a compound that decreases trimerization of CD40R, a compound that decreases trimerization of CD40L, a compound that modulates the CD40L/CD40R signaling pathway upstream or downstream of CD40L/CD40R interaction, a compound that reduces the phosphorylation of the tau protein or mutants thereof, a compound that interferes with TNF receptor-associated factors, a compound that interferes with presenilin-1 and/or presenilin-2, a compound that inhibits β-secretase activity, a compound that inhibits γ-secretase activity, a compound that enhances α-secretase activity, a compound that alters APP processing, a compound that reduces the ratio of APP β-CTF to APP α-CTF, a compound that reduces the amount of β-CTF, a compound that promotes brain-to-blood clearance of β-amyloid, a compound that increases circulating levels of β-amyloid, a compound that reduces the size and/or number of amyloid plaques, a compound that reduces β-amyloid burden, a compound that reduces soluble β-amyloid levels, a compound that reduces total β-amyloid levels, a compound that reduces congophilic β-amyloid deposits, a compound that reduces reactive gliosis, microgliosis, astrocytosis, and combinations thereof, and a compound selected from the group consisting of CD40L, soluble CD40R, soluble CD40L, immunogenic CD40L, CD40L variants (CD40LV), an antibody that binds to CD40L and blocks its interaction with CD40R, an antibody that binds with CD40R and blocks ligand binding to CD40R, a soluble CD40LV compound that binds to CD40R and fails to activate CD40R, an interfering RNA (dsRNA, RNAi or siRNA) compound to CD40R, an antisense RNA compound to CD40R, an interfering RNA (dsRNA, RNAi or siRNA) compound to CD40L, an antisense RNA compound to CD40L, or combinations of interfering RNA (dsRNA, RNAi or siRNA) compounds and antisense compounds.

56. The method of claim 55, wherein the interfering RNA (dsRNA, RNAi or siRNA) comprises polynucleotide sequences identical or homologous to CD40L, CD40R or β-amyloid.

57. The method of claim 56, wherein the interfering RNA (dsRNA, RNAi or siRNA) has greater than 70% homology to CD40L, CD40R or β-amyloid.

58. The method of claim 56, wherein the interfering RNA (dsRNA, RNAi or siRNA) has greater than 95% homology to CD40L, CD40R or β-amyloid.

59. The method of claim 55, wherein gene fragments of CD40L, CD40R or β-amyloid are targeted with the interfering RNA (dsRNA, RNAi or siRNA), and wherein the gene fragments are approximately 15-25 nucleotides in length.

60. The method of claim 55, wherein gene fragments of CD40L, CD40R or β-amyloid are targeted with the interfering RNA (dsRNA, RNAi or siRNA), and wherein the gene fragments are greater than 25 nucleotides in length.

61. The method of claim 55, wherein gene fragments of CD40L, CD40R or β-amyloid are targeted with the interfering RNA (dsRNA, RNAi or siRNA), and wherein the gene fragments are greater than 50 nucleotides in length.

62. The method of claim 55, wherein gene fragments of CD40L, CD40R or β-amyloid are targeted with the interfering RNA (dsRNA, RNAi or siRNA), and wherein the gene fragments are one nucleotide less than the full length gene of CD40L, CD40R or β-amyloid.

63. The method of claim 62, wherein the amyloidogenic disease is selected from the group consisting of Alzheimer's disease, scrapie, transmissible spongiform encepalopathy, hereditary cerebral hemorrhage with amyloidosis Icelandic-type, hereditary cerebral hemorrhage with amyloidosis Dutch-type, familial Mediterranean fever, familial amyloid nephropathy with urticaria and deafness (Muckle-Wells syndrome), myeloma or macroglobulinemia-associated idiopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes Type II, and insulinoma.

64. The method of claim 62, wherein the tauopathy is selected from the group consisting of Alzheimer's disease, frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amyotrophy complex, Pick's disease, and Pick's disease-like dementia.

65. The method of claim 62, wherein the carrier is a pharmaceutically acceptable carrier or diluent.

66. The method of claim 62, wherein the route of administration of the composition to the individual is via parenteral, oral or intraperitoneal administration.

67. The method of claim 66, wherein the parenteral route of administration is selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, and nasal inhalation via insufflation or nebulization.

68. The method of claim 66, wherein the composition of the carrier and the agent is administered orally in the form of hard or soft shell gelatin capsules, tablets, troches, sachets, lozenges, elixirs, suspensions, syrups, wafers, powders, granules, solutions or emulsions.

69. The method of claim 66, wherein the nasal administration of the composition of the carrier and the agent is selected from the group consisting of aerosols, atomizers and nebulizers.

70. The method of claim 66, further comprising administering the therapeutically effective amount of the composition of the agent and a carrier with other therapeutically effective compositions simultaneously or in intervals.

71. A method of causing a desired biological effect in an animal, human or system afflicted with neuronal inflammation, brain injury, brain trauma, a tauopathy, or an amyloidogenic disease, comprising the administration of a composition comprising a carrier and an agent that interferes with the CD40L/CD40R signaling pathway of the individual or system in amounts sufficient to cause the desired biological effect.

72. The method of claim 71, wherein the desired biological effect is a decrease in neuronal inflammation, a decrease in the trimerization of CD40R, a decrease in the trimerization of CD40L, modulation of the CD40L/CD40R signaling pathway upstream or downstream of CD40L/CD40R interaction, reduction in the phosphorylation of the tau protein or mutants thereof, interference with TNF receptor-associated factors, interference with presenilin-1 and/or presenilin-2, inhibition of β-secretase activity, inhibition of γ-secretase activity, enhancement of α-secretase activity, alteration of APP processing, reduction in the ratio of APP β-CTF to APP α-CTF, reduction in the amount of β-CTF, promotion of brain-to-blood clearance of β-amyloid, an increase in the circulating levels of β-amyloid, a decrease in β-amyloid levels in the central nervous system, reduction in the size and/or number of amyloid plaques, reduction of β-amyloid burden, reduction of soluble β-amyloid levels, reduction of total β-amyloid levels, reduction of congophilic β-amyloid deposits, reduction of reactive gliosis, reduction in microgliosis, reduction in astrocytosis, or any combination of the above-described biological effects.

73. The method of claim 71, wherein the amyloidogenic disease is selected from the group consisting of Alzheimer's disease, scrapie, transmissible spongiform encepalopathy, hereditary cerebral hemorrhage with amyloidosis Icelandic-type, hereditary cerebral hemorrhage with amyloidosis Dutch-type, familial Mediterranean fever, familial amyloid nephropathy with urticaria and deafniess (Muckle-Wells syndrome), myeloma or macroglobulinemia-associated idiopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes Type II, and insulinoma.

74. The method of claim 71, wherein the tauopathy is selected from the group consisting of Alzheimer's disease, frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amyotrophy complex, Pick's disease, and Pick's disease-like dementia.

75. The method of claim 71, wherein the agent is a compound that binds to CD40R, a compound that binds to CD40L, a compound that decreases trimerization of CD40R, a compound that decreases trimerization of CD40L, a compound that modulates the CD40L/CD40R signaling pathway upstream or downstream of CD40L/CD40R interaction, a compound that reduces the phosphorylation of the tau protein or mutants thereof, a compound that interferes with TNF receptor-associated factors, a compound that interferes with presenilin-1 and/or presenilin-2, a compound that inhibits β-secretase activity, a compound that inhibits γ-secretase activity, a compound that enhances α-secretase activity, a compound that alters APP processing, a compound that reduces the ratio of APP β-CTF to APP α-CTF, a compound that reduces the amount of β-CTF, a compound that promotes brain-to-blood clearance of β-amyloid, a compound that increases circulating levels of β-amyloid, a compound that reduces the size and/or number of amyloid plaques, a compound that reduces β-amyloid burden, a compound that reduces soluble β-amyloid levels, a compound that reduces total β-amyloid levels, a compound that reduces congophilic β-amyloid deposits, a compound that reduces reactive gliosis, microgliosis, astrocytosis, and combinations thereof, and a compound selected from the group consisting of CD40R, CD40L, soluble CD40L, immunogenic CD40L, CD40L variants (CD40LV), antibodies that bind to CD40L and block its interaction with CD40R, antibodies that bind with CD40R and block ligand binding to CD40R, soluble CD40LV that bind to CD40R and fail to activate CD40R, interfering RNA (dsRNA, RNAi or siRNA) to CD40R, antisense RNA to CD40R, interfering RNA (dsRNA, RNAi or siRNA) to CD40L, antisense RNA to CD40L, and combinations thereof.

76. The method of claim 75, wherein the interfering RNA (dsRNA, RNAi or siRNA) comprises polynucleotide sequences identical or homologous to CD40L, CD40R or β-amyloid.

77. The method of claim 76, wherein the interfering RNA (dsRNA, RNAi or siRNA) has greater than 70% homology to CD40L, CD40R or β-amyloid.

78. The method of claim 76, wherein the interfering RNA (dsRNA, RNAi or siRNA) has greater than 95% homology to CD40L, CD40R or β-amyloid.

79. The method of claim 75, wherein gene fragments of CD40L, CD40R or β-amyloid are targeted with the interfering RNA (dsRNA, RNAi or siRNA), and wherein the gene fragments are approximately 15-25 nucleotides in length.

80. The method of claim 75, wherein gene fragments of CD40L, CD40R or β-amyloid are targeted with the interfering RNA (dsRNA, RNAi or siRNA), and wherein the gene fragments are greater than 25 nucleotides in length.

81. The method of claim 75, wherein gene fragments of CD40L, CD40R or β-amyloid are targeted with the interfering RNA (dsRNA, RNAi or siRNA), and wherein the gene fragments are greater than 50 nucleotides in length.

82. The method of claim 75, wherein gene fragments of CD40L, CD40R or β-amyloid are targeted with the interfering RNA (dsRNA, RNAi or siRNA), and wherein the gene fragments are one nucleotide less than the full length gene of CD40L, CD40R or β-amyloid.

83. The method of claim 71, wherein the agent is an anti-CD40R antibody.

84. The method of claim 80, wherein the anti-CD40R antibody is one or more species of monoclonal anti-CD40R antibodies, polyclonal antibodies to CD40R, or a combination of polyclonal and monclonal antibodies.

85. The method of claim 71, wherein the agent is an anti-CD40L antibody.

86. The method of claim 85, wherein the anti-CD40L antibody is one or more species of monoclonal anti-CD40L antibodies, polyclonal antibodies to CD40L, or a combination of polyclonal and monclonal antibodies.

87. The method of claim 71, wherein the carrier is a pharmaceutically acceptable carrier or diluent.

88. The method of claim 71, wherein the route of administration of the composition to the individual is via parenteral, oral or intraperitoneal administration.

89. The method of claim 88, wherein the parenteral route of administration is selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, and nasal inhalation via insufflation or nebulization.

90. The method of claim 88, wherein the composition of the carrier and the agent is administered orally in the form of hard or soft shell gelatin capsules, tablets, troches, sachets, lozenges, elixirs, suspensions, syrups, wafers, powders, granules, solutions or emulsions.

91. The method of claim 89, wherein the nasal administration of the composition of the carrier and the agent is selected from the group consisting of aerosols, atomizers and nebulizers.

92. The method of claim 71, further comprising administering the therapeutically effective amount of the composition of the agent and a carrier with other therapeutically effective compositions simultaneously or in intervals.
